(19) United States
(12) Patent Application Publication
(10) Pub. No.: US 2025/0154455 A1
(43) Pub. Date: May 15, 2025

(54) BIOACTIVE AND ANTIOXIDANT SUPRAMOLECULAR POLYMER HYDROGELS FOR NEURAL CELL CULTURE

(71) Applicant: Northwestern University, Evanston, IL (US)

(72) Inventors: Nicholas A. Sather, Evanston, IL (US); Bo Timmy Bjoern Fyrner, Evanston, IL (US); Samuel Isaac Stupp, Evanston, IL (US); Anna Metlushko, Evanston, IL (US)

(21) Appl. No.: 18/941,862

(22) Filed: Nov. 8, 2024

Related U.S. Application Data
(60) Provisional application No. 63/548,029, filed on Nov. 10, 2023.

(51) Int. Cl. C12N 5/0793; C12M 1/12

(52) U.S. Cl. CPC C12N 5/0619; C12M 25/02; C12N 2513/00; C12N 2533/30; C12N 2533/50; C12N 2537/10

(57) ABSTRACT

Provided herein are compositions comprising a copolymer comprising at least one sulfonatoalkoxy EDOT monomer (e.g. EDOT-S) or at least one cationic EDOT monomer and at least one heterocyclic monomer (e.g. an EDOT-OH monomer) and one or more bioactive peptide amphiphiles (PAs). The compositions provided herein find use in cell culture methods and in methods of promoting neural growth and maturation.

### PRIORITY STATEMENT

This application claims priority to U.S. Provisional Application No. 63/548,029, filed Nov. 10, 2023, the entire contents of which are incorporated herein by reference for all purposes.

### SEQUENCE LISTING

The text of the computer readable sequence listing filed herewith, titled “NWEST-42569-202_SQL.xml”, created Nov. 8, 2024, having a file size of 10,560 bytes, is hereby incorporated by reference in its entirety.

### FIELD

Provided herein are compositions comprising a conductive copolymer comprising at least one sulfonatoalkoxy EDOT monomer or at least one cationic EDOT monomer, and at least one heterocyclic monomer, and uses thereof. In some aspects, provided herein are compositions comprising at least one EDOT-S monomer and at least one EDOT-OH monomer and one or more bioactive peptide amphiphiles (PAs). The compositions provided herein find use in cell culture methods and in methods of promoting neural growth and maturation.

### BACKGROUND

Potential treatments to enhance CNS regeneration include cell and therapeutic delivery to promote or direct neuron growth. Cells delivered to the injured site can directly replace damaged neurons and secrete neurotrophic factors to promote regeneration of native cells. Unfortunately, the majority of transplanted cells die shortly after injection without the presence of a supporting scaffold. Moreover, protein growth factors can also be delivered to the injury site to aid native cells in their axon regeneration into the glial scar. However, these proteins often diffuse into the surrounding area and result in short term regeneration. Accordingly, what is needed are improved methods for delivering therapies to the central nervous system or to cells derived therefrom, including for treatment of disease.

### SUMMARY

In some aspects, provided herein are conductive copolymers and uses thereof. In some embodiments, provided herein are copolymers comprising at least one sulfonatoalkoxy EDOT monomer or at least one cationic EDOT monomer and at least one heterocyclic monomer. In some embodiments, the compositions comprise least one EDOT-S monomer and at least one heterocyclic monomer. In some embodiments, the compositions comprise at least one cationic EDOT monomer and at least one heterocyclic monomer. In some embodiments, the copolymers provided herein comprise a plurality of heterocyclic monomers. In some embodiments, each heterocyclic monomer comprises at least one aromatic ring.

In some embodiments, the at least one heterocyclic monomer comprises a thiophene monomer. In some embodiments, the at least one heterocyclic monomer comprises at least one one EDOT monomer. In some embodiments, the at least one EDOT monomer is functionalized. In some embodiments, the at least one EDOT monomer is functionalized with a hydroxyl group or a methylimidazole group. The functional group (e.g. —OH or methylimidazole) may be attached to the EDOT monomer by a suitable linker. In some embodiments, the functional group is attached covalently. In some embodiments, the copolymer comprises four EDOT-s monomers and one EDOT monomer containing a suitable functional group.

In some embodiments, the copolymer comprises the at least one sulfonatoalkoxy EDOT monomer or at least one cationic EDOT monomer and the at least one heterocyclic monomer at a ratio of 1:10 to 10:1. In some embodiments, the copolymer comprises the at least one sulfonatoalkoxy EDOT monomer or at least one cationic EDOT monomer and the at least one heterocyclic monomer at a ratio of 2:1 to 6:1. In some embodiments, the copolymer comprises the at least one sulfonatoalkoxy EDOT monomer or at least one cationic EDOT monomer and the at least one heterocyclic monomer at a ratio of 4:1.

In some embodiments, the copolymer comprises at least one EDOT-S monomer and least one EDOT-OH monomer. In some embodiments, the copolymer comprises four EDOT-S monomers and one EDOT-OH monomer. In some embodiments, provided herein are copolymers comprising a plurality of functionalized EDOT derivatives.

In some embodiments, the copolymers described herein are incorporated into compositions comprising a hydrogel material. In some embodiments, provided herein are compositions comprising a copolymer provided herein and a hydrogel material. In some embodiments, the composition further comprises a linker that covalently or noncovalently links the copolymer to the hydrogel material.

The composition may comprise any suitable hydrogel material. In some embodiments, the hydrogel material comprises a polysaccharide. In some embodiments, the hydrogel material comprises gellan gum. In some embodiments, the hydrogel material comprises a plurality of bioactive peptide amphiphiles, each bioactive peptide amphiphile comprising a hydrophobic tail, a structural peptide segment, a charged peptide segment, and a bioactive moiety (e.g., peptide, glycan). In some embodiments, the composition further comprises one or more filler peptide amphiphiles, wherein the filler peptide amphiphiles comprise a hydrophobic non-peptide tail, a structural peptide segment, and a charged peptide segment, and do not comprise a bioactive moiety. In some embodiments, the hydrogel material comprises gellan gum and a plurality of bioactive peptide amphiphiles.

The compositions provided herein may be used in cell culture methods and are shown herein to promote cell growth, maturation, and signaling. For example, inclusion of a copolymer provided herein (e.g. PEDOT-S/OH) in a composition provides a conductive composition while also, surprisingly, providing an antioxidant effect. This combination of an antioxidant effect, a conductive copolymer, and a bioactive peptide provides for compositions that effectively mimic the extracellular matrix electrically, physically, and chemically, and thus promote cell growth and maturation while minimizing the detrimental impacts of free radical generation. In some embodiments, provide herein is a method comprising contacting a cell with a composition provided herein. In some embodiments, provided herein are systems comprising a cell cultured on a composition provided herein. In some embodiments, the compositions provided herein are used to promote growth, differentiation, and/or activity of a cell, including an electrogenic cell. In some embodiments, the cell is a neuron. In some embodiments, the cell is a muscle cell. For example, in some embodiments the cell is a cardiac cell.

In some embodiments, provided herein is a method comprising providing a composition or a system provided herein to a subject. In some embodiments, the subject has a nervous system injury, and the composition is provided to the subject to promote cell growth, regeneration, or healing from the injury.

### BRIEF DESCRIPTION OF THE DRAWINGS

FIGS. 1A-1C show PEDOT-S leakage and cytotoxicity in hydrogel scaffolds. FIG. 1A shows the chemical structure of Gellan Gum (GG) and PEDOT-S. FIG. 1B shows brightfield images of C2C12 cells cultured on GG and GG+PEDOT-S. FIG. 1C shows photographs of GG and PEDOT-S+GG gels showing leakage of PEDOT-S into solution over the course of 7 days.

FIGS. 2A-2E show PEDOT-S/OH shows more stable gels and enhanced biocompatibility. FIG. 2A shows chemical structures of Gellan Gum (GG), PEDOT-S, and PEDOT-S/OH. FIG. 2B shows photographs of GG gels with either PEDOT-S and PEDOT-S/OH after 1 day and 7 days of soaking in saline. FIG. 2C is a graph showing leakage from absorbance spectroscopy over the course of 120 hours. FIG. 2D shows quantitative leakage analysis of PEDOT+GG gels from absorbance spectroscopy at 824 nm. FIG. 2E shows fluorescence micrographs of C2C12 cells stained with live (calcein AM) and dead (Eh-1) markers. The bar graph shows viability quantification from live/dead staining.

FIG. 3 shows Elemental analysis of PEDOT-S/OH copolymer, consistent with 4:1 ratio of EDOT-S to EDOT-OH monomers.

FIGS. 4A-4C show Physical characterization of GG and PEDOT hydrogels. FIG. 4A shows electrical impedance spectroscopy at 10 mV perturbation. FIG. 4B shows chronoamperometry at 100 mV direct current. FIG. 4C shows storage modulus of gels at 0.1% strain.

FIGS. 5A-5B show absorbance spectroscopy and PEDOT leakage of gels. FIG. 5A shows absorbance spectroscopy of GG+PEDOT gels with and without DVS crosslinker. FIG. 5B shows PEDOT leakage quantification from absorbance at 824 nm.

FIGS. 6A-6L show enhanced neural maturation on PA+PEDOT-S/OH coatings. FIG. 6A shows the chemical structure of IKVAV PA. FIG. 6B shows biocompatibility of cortical neurons on PA+PEDOT coatings. Images show live/dead staining of cortical neurons, the bar graph shows live/dead image quantification. FIG. 6C shows widefield, confocal and SEM imaging of cortical neurons on PA+PEDOT coatings. FIG. 6D shows quantification of neurite length from brightfield imaging. FIG. 6E-6H show western blot (FIG. 6E) and quantification at DIV5 of maturation and synaptic markers MAP-2 (FIG. 6F), PSD-95 (FIG. 6G), and synaptophysin (FIG. 6H) from cortical neuron protein extract. FIGS. 6I-6J show patch clamp electrophysiology of cortical neurons (FIG. 6I) with quantitative analysis of action potential amplitude (FIG. 6J) and membrane resting potential (FIG. 6K). FIG. 6L shows RNA gene ontology of upregulated pathways in PA+PEDOT-S/OH compared to PA alone.

FIGS. 7A-7D show morphological analysis of cortical neuron growth. FIG. 7A shows Sholl analysis of DIV 3 cortical neurons growth on PA+PEDOT coatings. FIG. 7B and FIG. 7C shows time course of cortical neuron growth on PA+PEDOT coatings with quantification of neurite length (FIG. 7B) and number of branches (FIG. 7C). FIG. 7D shows position analysis of soma migration over time. The bar graph shows quantification of soma velocity.

FIGS. 8A-8B show cortical neuron maturation on control PA coatings. E2 PA is a non-bioactive molecule that lacks the IKVAV (SEQ ID NO: 1) epitope and the V2A2 IKVAV PA has a mutation in the peptide sequence that reduces the availability of the IKVAV (SEQ ID NO: 1) epitope for signaling. FIG. 8A shows widefield fluorescence imaging of cortical neurons grown on PA+PEDOT coatings. FIG. 8B shows corresponding neurite length analysis from images in (a).

FIGS. 9A-9I show mechanisms for enhanced maturation in PEDOT-S/OH coatings. FIG. 9A shows confocal micrographs of cortical neurons grown on IKVAV PA coatings with and without PEDOT-S/OH. FIG. 9B shows intensity quantification of pCREB/DAPI from images in (a). FIG. 9C shows western blot of CREB and pCREB expression. FIG. 9D shows quantification of pCREB expression in (c). FIG. 9E shows ROS quantification in cell media of cortical neuron cultures grown on IKVAV PA with and without PEDOT-S/OH. FIG. 9F shows remaining hydrogen peroxide detected in solutions of PA or PEDOT-S/OH over time. FIG. 9G shows brightfield micrographs of cortical neurons cultured on PA with and without PS2, with fluorescence signal from intracellular ROS overlayed. FIG. 9H shows quantification of ROS⁺ cells after 24 h FIG. 9I shows fluorescence intensity of ROS in cortical neurons. FIG. 9I shows western blot and quantification of pP38 and P38 expression (n=3, p<0.05).

FIGS. 10A-10O show possible maturation pathways. FIG. 10A, FIG. 10B, FIG. 10C, and FIG. 10D show western blot and quantification of the pERK/ERK pathway. FIG. 10E, FIG. 10F, FIG. 10G, and FIG. 10H show western blot and quantification of PKA expression. FIG. 10G and FIG. 10H show western blot and quantification of calcineurin expression. FIG. 10I, FIG. 10J, and FIG. 10K show western blot and quantification of PI3K expression. FIG. 10L, FIG. 10M, FIG. 10N, and FIG. 10O show western blot and quantification of the pCAMKII/CAMKII pathway.

FIGS. 11A-11F show applications of conducting polymer-supramolecular polymer hydrogels. FIG. 11A shows confocal micrographs of human iPSC-derived neural progenitor cells (Nestin, green) maturing into neurons (Tuj1, red) when cultured on PA+PEDOT-S/OH coatings. FIG. 11B shows quantification of neuron content in hiPSCs culture from immunostaining in (a). FIG. 11C shows confocal micrographs of human spinal cord organoids treated with PA alone or PA+PEDOT-S/OH in solution. FIG. 11D shows average neurite length sprouting from organoid surface from immunostaining in (c). FIG. 11E and FIG. 11F shows photographs of 3D printed Northwestern University logo and anatomical nose using GG+PA+PEDOT-S/OH hydrogel inks.

FIG. 12 shows exemplary heterocyclic monomers synthesized herein. Such heterocyclic monomers may be used in a composition provided herein.

FIG. 13 is a schematic molecular drawing demonstrating the copolymer properties can be tuned by introducing different monomer depicted (RED). The X atom includes but not limited to nitrogen (N), oxygen (O), sulfur (S), or phosphorous (P).

FIG. 14A shows ¹H HRMAS NMR spectra of PS1, PS2, GG, GG+PS1, and GG+PS2 highlighting the —OH region. FIG. 14B shows size measurements of PS1 and PS2 particles from dynamic light scattering.

FIG. 15A is a schematic of printing extruded hydrogel filaments with inset showing depiction of aligned fibers along printing direction. FIG. 15B shows polarized optical microscopy images of printed GG+PS2 and GG+PA+PS2 filaments. FIG. 15C shows quantification of birefringence intensity using the ratio of transmitted light intensity with the filament oriented 450 vs. 0° to the polarizer. FIG. 15D is a schematic of mouse cortical neurons seeded on printed filaments after 1 week of culture. FIG. 15E shows confocal micrographs of murine cortical neurons on printed filaments (TUJ1, green). FIG. 15F is a plot of the distribution of neuron orientation on printed filaments relative to printing direction from confocal micrographs (n=3, p<0.05).

FIG. 16 shows cationic EDOT derivatives with extended linker moieties.

FIG. 17 shows chemical structures of PEDOT-Bu-Im/OH copolymer and PEDOT-EG2-Im/OH copolymer.

FIG. 18A-18B shows purity measurement using Liquid chromatography-mass spectrometry (LCMS). FIG. 18A shows LCMS chromatograms and corresponding mass spectra of 3-(4-((2,3-dihydrothieno[3,4-b][1,4]dioxi-2-yl)methoxy)butyl)-1-methyl-1H-imidazol-3-ium chloride (EDOT-Bu-ImCl). FIG. 18B shows LCMS chromatograms and corresponding mass spectra of 3-(2-(2-((2,3-dihydrothieno[3,4-b][1,4]dioxin-2-yl)methoxy)ethoxy)ethyl)-1-methyl-1H-imidazol-3-ium chloride (EDOT-EG2-ImCl).

FIGS. 19A-19N show nuclear magnetic resonance (NMR) analysis for synthesized compounds. FIG. 19A shows ¹H-NMR spectra in CDCl₃ of 4-(trityloxy)butyl 4-methylbenzenesulfonate (TrO-Bu-OTs). FIG. 19A shows ¹³C-NMR spectra in CDCl₃ of 4-(trityloxy)butyl 4-methylbenzenesulfonate (TrO-Bu-OTs).

### DETAILED DESCRIPTION

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, the term “peptoid” refers to a class of peptidomimetics where the side chains are functionalized on the nitrogen atom of the peptide backbone rather than to the α-carbon.

As used herein, the term “bioisostere” refers to chemical substituents or functional groups that exhibit similar physicochemical properties and produce similar biological properties as the native functional group being mimicked. “Classical bioisosteres” are functional groups categorized as: monovalent atoms or groups, divalent atoms or groups, trivalent atoms or groups, tetravalent atoms and groups, and ring equivalents. “Non-classical bioisosteres” are counterparts that do not fulfill the steric and electronic criteria required for the “classical bioisosteres”.

Examples of bioisosteres, are but not limited to: hydrogen and fluoride, deuterium and hydrogens, carboxylic acid and the tetrazole ring, carboxylic acid and acyl sulfonamide, carboxylic acid and aryl sulfonamide, amide and imidazole, amide and ester, amide and carbamate, amide and urea.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “nanofiber” refers to an elongated or threadlike filament (e.g., having a significantly greater length dimension that width or diameter) with a diameter typically less than 100 nanometers.

As used herein, the term “scaffold” refers to a material capable of supporting growth and differentiation of a cell.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide lipophilic (hydrophobic) segment, a structural peptide segment and/or charged peptide segment (often both), and optionally a bioactive segment (e.g., linker segment, bioactive segment, etc.). The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment; (3) a charged peptide segment, and (4) a bioactive segment (e.g., linker segment).

As used herein and in the appended claims, the term “lipophilic moiety” or “hydrophobic moiety” refers to the moiety (e.g., an acyl, ether, sulfonamide, or phosphodiester moiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)— where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used interchangeably herein, the terms “structural peptide” or “structural peptide segment” refer to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural peptide segments of adjacent structural peptide segments. In some embodiments, nanofibers of peptide amphiphiles having structural peptide segments display linear or 2D structure when examined by microscopy and/or α-helix and/or β-sheet character when examined by circular dichroism (CD). In some embodiments, nanofibers of peptide amphiphiles having structural peptide segments with a total propensity for forming β-sheet conformations of 4 or less display a less ordered character (e.g. less ordered secondary structure, such as less rigid β-sheet conformations). In some embodiments, nanofibers of peptide amphiphiles having structural peptide segments with a total propensity for forming β-sheet conformations of 4 or less (e.g. A₂G₂) display a propensity to form random coil structures.

As used herein, the term “beta (β)-sheet-forming peptide segment” refers to a structural peptide segment that has a propensity to display β-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises one or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, and or about 3 or 4 amino acids in this segment.

As used herein, the terms “amino-rich peptide segment”, “basic peptide segment,” and “positively-charged peptide segment” refer to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A basic peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., an IKVAV peptide) exhibit the functionality of the bioactive peptide.

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the term “heterocyclic monomer” relates to a monomer consisting of a heterocycle that can be used to form a polymer, either as a homopolymer using the same monomer, or as copolymer with different monomers at various ratios.

As used herein, the term “heterocycles” relates to a type of organic compound that contains a ring structure made up of carbon atoms and at least one atom of a different element, typically nitrogen (N), oxygen (O), sulfur (S), or phosphorous (P) within the ring. Heterocycles can be of different e.g., five-, six-, or seven-membered rings. Examples of heterocycles include but are not limited to thiophene, furan, pyrrole, imidazole, triazole, tetrazole, pyridine. Heterocycles can consist of a bicyclic structure where rings are fused together. Examples of fused bicyclic heterocyles are, but not limited to, indoles and indolizines.

As used herein, the term “derivative” relates to a chemical compound that originates from another chemical compound as a result of a chemical reaction or alteration. A “derivative” is often created by, but not limited to, making specific changes to the structure of a parent compound, such as adding functional groups or substituting atoms or groups of atoms.

As used herein, the term “EDOT” or “EDOT monomer” relates to the chemical structure 3,4-ethylenedioxythiophene.

As used herein, the terms “treat,” “treatment,” and “treating” refer to reducing the amount or severity of a particular condition, disease state (e.g., CNS injury), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular condition or disease state (e.g., CNS injury) from occurring in a subject not presently experiencing or afflicted with the condition or disease state. The terms do not necessarily indicate complete or absolute prevention. For example, “preventing CNS injury” refers to reducing the likelihood of CNS injury occurring in a subject not presently experiencing or diagnosed with a CNS injury. In order to “prevent CNS injury” a composition or method need only reduce the likelihood of CNS injury, not completely block any possibility thereof. “Prevention,” encompasses any administration or application of a therapeutic or technique to reduce the likelihood of a disease developing (e.g., in a mammal, including a human). Such a likelihood may be assessed for a population or for an individual.

As used herein, the terms “co-administration” and “co-administering” refer to the administration of at least two agent(s) or therapies to a subject (e.g., an IKVAV PA nanofiber and one or more therapeutic agents). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Biomaterial scaffolds are used as a physical support vehicle for the delivery of cells, proteins, and pharmaceuticals and a scaffold for regenerating native cells. Incorporating an electroactive component into a biomaterial scaffold potentially allows for the propagation of electrical signals between neurons cultured on the scaffold and the surrounding tissue, furthering their communication and regeneration. A promising conducting polymer is poly(3,4-ethylenedioxythiophene) (PEDOT). This material is commercially available and has been utilized as an electrode coating and flexible electronic substrate. PEDOT polymerization is usually conducted in the presence of an anion, such as poly(styrene sulfonate) (PSS), which stabilizes the radical cations on the conjugated backbone, resulting in a conductive polyelectrolyte complex (PEDOT:PSS). PEDOT is believed to polymerize onto the PSS backbone, resulting in an aqueous dispersion of PEDOT grains surrounded by a shell of excess PSS. PEDOT:PSS possesses high conductivity (>1,000 S cm⁻¹) and chemical stability. The material can be easily processed into coatings and printed, showing its versatility and potential for tissue engineering and cell culture models. However, PSS is insulative, limits cell adhesion, and has potentially toxic metabolic products, leading to the search for alternative ways to “dope” (i.e., stabilize radical cations) in PEDOT without PSS. Recent advances have focused on chemically altering the structure of PEDOT itself to eliminate the PSS and create a “self-doped” and more biocompatible conducting polymer. PEDOT-S, which contains a pendant sulfonate group attached to the thiophene backbone, has been synthesized. However, due to the increased water solubility of PEDOT-S, it can leak out of hydrogels when placed in cell media. This results in potential cell membrane permeability and reduced biocompatibility. Thus, there is a need for further functionalization of the PEDOT-S structure to address the leakage issue and reduce cytotoxicity. The present disclosure addresses this need and provides a copolymer suitable for use in biomaterial scaffolds.

In some aspects, provided herein is a copolymer comprising (i) a sulfonatoalkoxy EDOT monomer or (ii) at least one cationic EDOT monomer, and at least one heterocyclic monomer. Such a copolymer is also referred to herein as a “conductive copolymer” or a “conducting copolymer”. In some embodiments, the sulfonatoalkoxy EDOT monomer is anionic. In some embodiments, the sulfonatoalkoxy EDOT monomer is cationic. EDOT refers to 3,4-ethylenedioxythiophene, the structure of which is shown below:

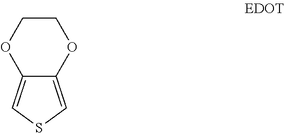

In some embodiments, the sulfonatoalkoxy EDOT monomer is the sulfonatoalkoxy EDOT derivative EDOT-S. In some embodiments, the sulfonatoalkoxy EDOT monomer is the branched sulfonatoalkoxy EDOT derivative S-EDOT. The term “sulfonatoalkoxy EDOT monomer” is inclusive of both S-EDOT and EDOT-S. The structures of S-EDOT and EDOT-S are as follows:

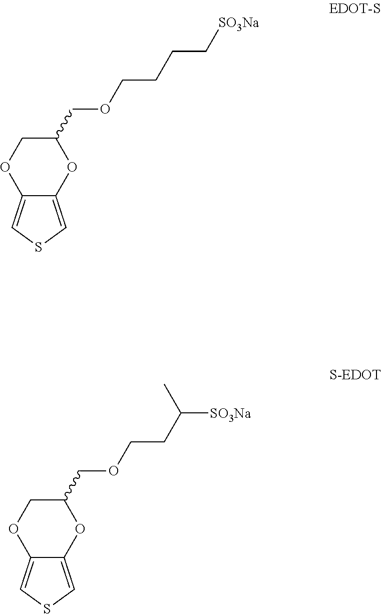

Although the copolymers herein are frequently referred to as comprising EDOT-S or at least one EDOT-S monomer, it is understood that S-EDOT or at least one S-EDOT monomer may alternatively be used as the sulfonatoalkoxy EDOT monomer.

In some embodiments, the copolymer comprises PEDOT-S.

The structure of PEDOT-S is as follows:

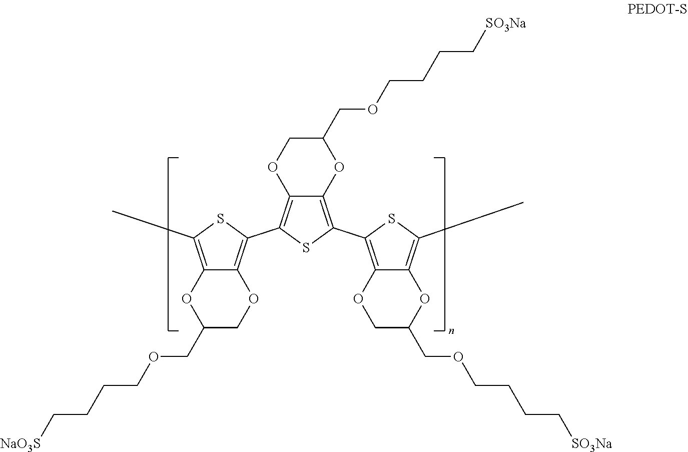

In some embodiments, the sulfonatoalkoxy EDOT monomer is an anionic sulfonatoalkoxy EDOT monomer. The term “anionic” indicates that the monomer is functionalized with an anionic (e.g. negatively charged) functional group. In some embodiments, the anionic functional group is attached to a carbon in the dioxane ring of EDOT. The anionic functional group may be attached to the EDOT monomer by any suitable linker. In some embodiments, the anionic functional group is a sulfonate group. In some embodiments, the copolymer comprises a cationic EDOT sulfonatoalkoxy monomer and at least one heterocyclic monomer. The term “cationic” indicates that the monomer is functionalized with a cationic (e.g. positively charged) functional group.

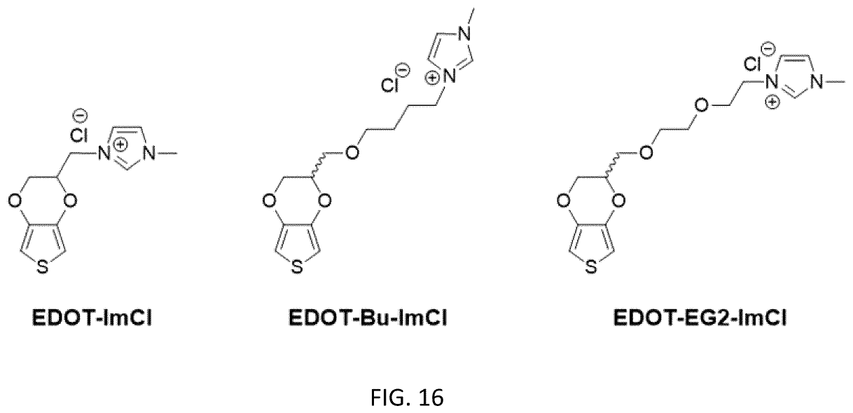

In some embodiments, the composition comprises at least one cationic EDOT monomer and at least one heterocyclic monomer. The term “cationic EDOT monomer” refers to an EDOT monomer functionalized with a cationic (e.g. positively charged) functional group. Such cationic EDOT monomers may attract anions in tissue, which may assist in use in neural repair. In some embodiments, the cationic functional group is attached to a carbon in the dioxane ring of EDOT. The cationic functional group may be attached to the EDOT monomer by any suitable linker. Exemplary linkers are described in detail below. Any suitable cationic functional group may be used. Exemplary cationic functional groups are shown in FIG. 16. In some embodiment, the cationic functional group comprises an imidazolium ion. In some embodiment, the cationic functional group comprises an N-methyl imidazolium ion. In some embodiments, the imidazolium ion comprises an alkylene chain:

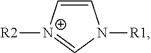

In some embodiments, the imidazolium ion comprises an N-alkylene chain wherein R1 is 1 to 20, 1 to 10, 1 to 5, or 1 to 3. For example, in some embodiments the imidazolium ion comprises an N-alkylene chain where R1 is 1, 2, 3, 4, or 5. For example, in some embodiments the imidazolium ion comprises an N-alkylene chain that is terminated by an —O—. In some embodiments, the imidazolium ion comprises a N-alkylated poly- or oligo-ethylene glycol chain.

In some embodiment the cationic functional group comprises a quaternary ammonium ion. In some embodiment, the quaternary ammonium ion comprises but not limited to alkylene chains:

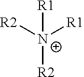

where R2 is attached to the EDOT-monomer. In some embodiments, R2 is a linker attached to the EDOT-monomer. In some embodiments, the quaternary ammonium ion comprises alkylene chains wherein R1 is 1 to 20, 1 to 10, 1 to 5, or 1 to 3. For example, in some embodiments the quaternary ammonium ion comprises alkylene chains where R1 is 1, 2, 3, 4, or 5. In some embodiment, the quaternary ammonium ion consists a trimethylated species.

In some embodiment, the cationic functional group comprises a pyrrolidinium ion. In some embodiment, the cationic functional group comprises a pyridinium ion. In some embodiment, the cationic functional group comprises a piperidinium ion. In some embodiment, the cationic functional group comprises a phosphonium ion. In some embodiment, the cationic functional group comprises a sulfonium ion.

In some embodiments, the cationic EDOT monomer is an EDOT-imidazolium monomer. An EDOT-imidazolium monomer refers to an EDOT monomer functionalized with an imidazolium group. In some embodiments, an EDOT-imidazolium monomer refers to an EDOT monomer having an imidazolium group attached to a carbon in the dioxane ring of EDOT. The imidazolium group may be attached to the EDOT monomer by a suitable linker. Exemplary structures of EDOT-imidazolium monomers are shown in FIG. 16. In some embodiments, the cationic EDOT monomer is EDOT-lmCl, EDOT-Bu-lmCl, or EDOT-EG2-lmCl, each of which are an EDOT-imidazolium monomer with the imidazolium group attached to the EDOT monomer by different linkers.

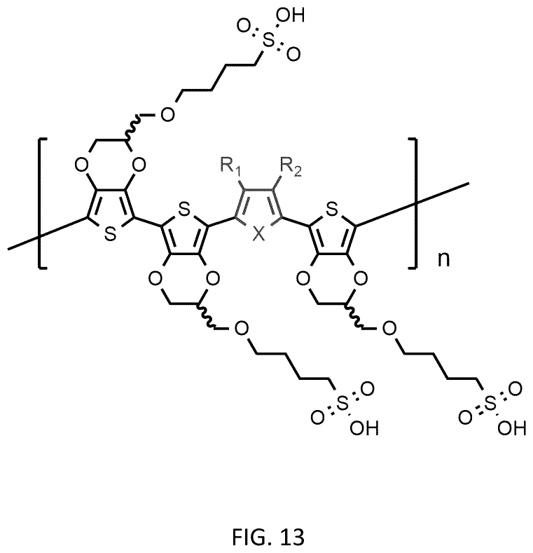

In some embodiments, the copolymer comprises PEDOT-S and the copolymer properties are tuned by using different heterocyclic monomers. For example, as shown in the schematic molecular drawing shown in FIG. 13, the copolymer properties can be tuned by introducing different heterocyclic monomers depicted as

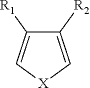

The X atom in the molecular drawing (FIG. 13) includes but not limited to nitrogen (N), oxygen (O), sulfur (S), or phosphorous (P). In some embodiments, the composition of the copolymer comprises one or more EDOT-S monomers and a thiophene monomer (R1═R2═H). R1 and R2 can be identical or different. R1 and/or R2 can be but not limited to alkoxy groups, substituted 3,4-ethylenedioxy groups, unsaturated hydrocarbons, saturated hydrocarbons, alcohols, peptides, carbohydrates, polyols, oligoethylene glycols, polyethylene glycols, substituted oligoethylene glycols, substituted polyethylene glycols. In some embodiments, R1 contains a terminal azide/azido group. In some embodiments, R2 contains a terminal azide/azido group. In some embodiments, R1 contains a terminal alkyne. In some embodiments, R2 contains a terminal alkyne. In some embodiments, R1 or R2 contains a terminal cyclooctyne. In some embodiments the copolymer is a sodium salt-form (Na). In some embodiments the copolymer is in an H-form.

In some embodiments, the heterocyclic monomer comprises an EDOT monomer functionalized with a suitable moiety. In some embodiments, provided herein is a copolymer comprising at least one EDOT-S monomer and at least one EDOT monomer functionalized with a suitable moiety (e.g. functional group). In some embodiments, a copolymer comprising at least one S-EDOT monomer and at least one EDOT monomer functionalized with a suitable moiety (e.g. functional group). In some embodiments, provided herein is a copolymer comprising at least one cationic EDOT monomer and at least one EDOT monomer functionalized with a suitable functional group.

In some embodiments, the at least one heterocyclic monomer is an EDOT derivative. In some embodiments, the at least one heterocyclic monomer comprises an EDOT monomer functionalized with a suitable moiety (e.g. a functional moiety, or a functional group). In some embodiments, the functional moiety is a hydroxyl group. In some embodiments, the functional moiety is a methylimidazole.

In some embodiments, the at least one heterocyclic monomer comprises an EDOT-OH monomer. In some embodiments, the copolymer comprises at least one EDOT-S monomer and at least one EDOT-OH monomer. In some embodiments, the copolymer comprises at least one S-EDOT monomer and at least one EDOT-OH monomer. In some embodiments, the copolymer comprises at least one cationic EDOT monomer and at least one EDOT-OH monomer. An EDOT-OH monomer refers to an EDOT monomer functionalized with a hydroxyl group. In some embodiments, an EDOT-OH monomer refers to an EDOT derivative having a hydroxyl group attached to a carbon in the dioxane ring of EDOT. The structure of EDOT-OH is shown below:

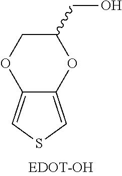

As used herein, the symbol “˜” represents a point of attachment to another moiety with unspecified stereochemistry at a chiral stereocenter. In some embodiments, the hydroxyl group is attached to the dioxane ring by a linker. In some embodiments, the hydroxyl group is attached to the linker covalently. In some embodiments, the hydroxyl group is attached to the linker and the linker is attached to the dioxane ring by covalent interactions. The linker may be any suitable length to facilitate the desired spacing of the hydroxyl group from the dioxane ring.

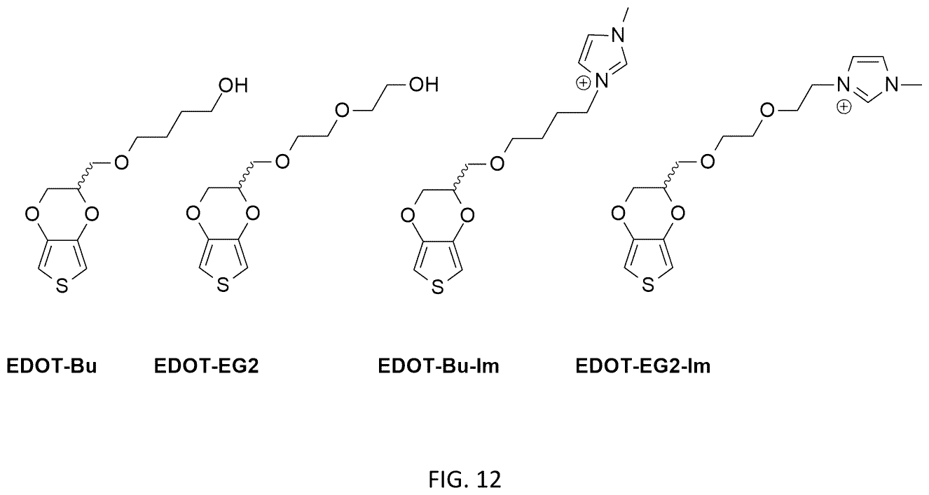

In some embodiments, the heterocyclic monomer is EDOT-Bu (an exemplary EDOT-OH monomer) or EDOT-EG2 (another exemplary EDOT-OH monomer), as shown in FIG. 12. EDOT-Bu an EDOT-EG2 are exemplary EDOT-OH monomers wherein the hydroxyl functional moiety is attached to the dioxane ring by a different linker.

In some embodiments, the at least one heterocyclic monomer is functionalized with a terminal methylimidazole. For example, in some embodiments the at least one heterocyclic monomer comprises an EDOT monomer functionalized with a terminal methylimidazole. The terminal methylimidazole may be attached to the dioxane ring in EDOT by any suitable linker.

In some embodiments, the copolymer comprises the at least one sulfonatoalkoxy EDOT monomer (e.g. EDOT-S monomers, S-EDOT monomers) and the at least one heterocyclic monomer (e.g. EDOT-OH) at a ratio of 1:10 to 10:1. For example, in some embodiments the copolymer comprises the at least one sulfonatoalkoxy EDOT monomer (e.g. EDOT-S monomers, S-EDOT monomers) and the at least one heterocyclic monomer (e.g. EDOT-OH) at a ratio of 1:10 to 10:1, 1:2 to 9:1, 1:1 to 8:1, 2:1 to 7:1, 3:1 to 6:1, or 4:1 to 5:1. In some embodiments, the copolymer comprises the at least one sulfonatoalkoxy EDOT monomer (e.g. EDOT-S monomers, S-EDOT monomers) and the at least one heterocyclic monomer (e.g. EDOT-OH) at a ratio of 1:10, 2:10, 3:10, 4:10, 1:2, 6:10, 7:10, 8:10, 9:10, 1:1, 2:1, 3:1, 4:1, 5:1, 6:1, 7:1, 8:1, 9:1 or 10:1. In some embodiments, the copolymer comprises the at least one sulfonatoalkoxy EDOT monomer (e.g. EDOT-S monomers, S-EDOT monomers) and the at least one heterocyclic monomer (e.g. EDOT-OH) at a ratio of 2:1 to 6:1. In some embodiments, the copolymer comprises the at least one sulfonatoalkoxy EDOT monomer (e.g. EDOT-S monomers, S-EDOT monomers) and the at least one heterocyclic monomer (e.g. EDOT-OH) at a ratio of 4:1.

In some embodiments, the copolymer comprises four EDOT-S monomers and one heterocyclic monomer. In some embodiments, the copolymer comprises four EDOT-S monomers and one EDOT derivative functionalized with a suitable moiety. In some embodiments, the copolymer comprises four EDOT-S monomers and one EDOT-OH monomer. In some embodiments, the copolymer consists of four EDOT-S monomers and one EDOT-OH monomer.

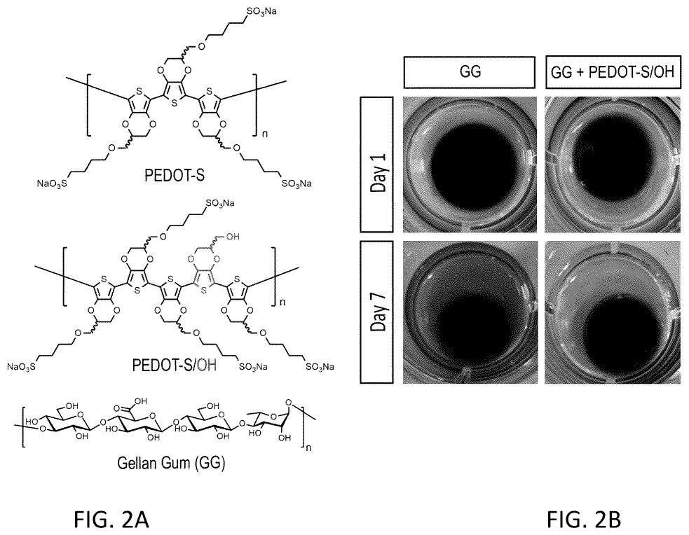

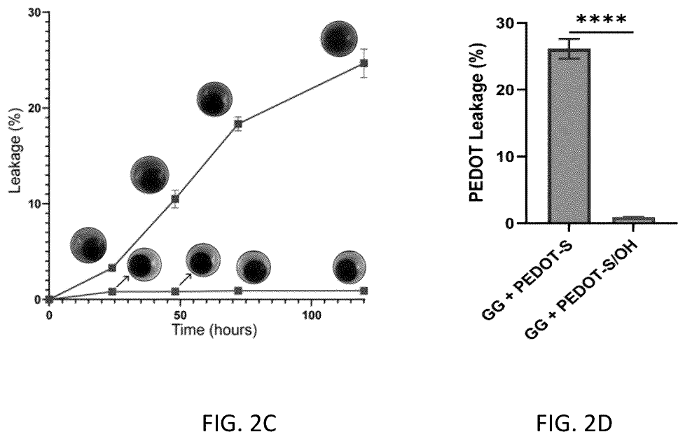

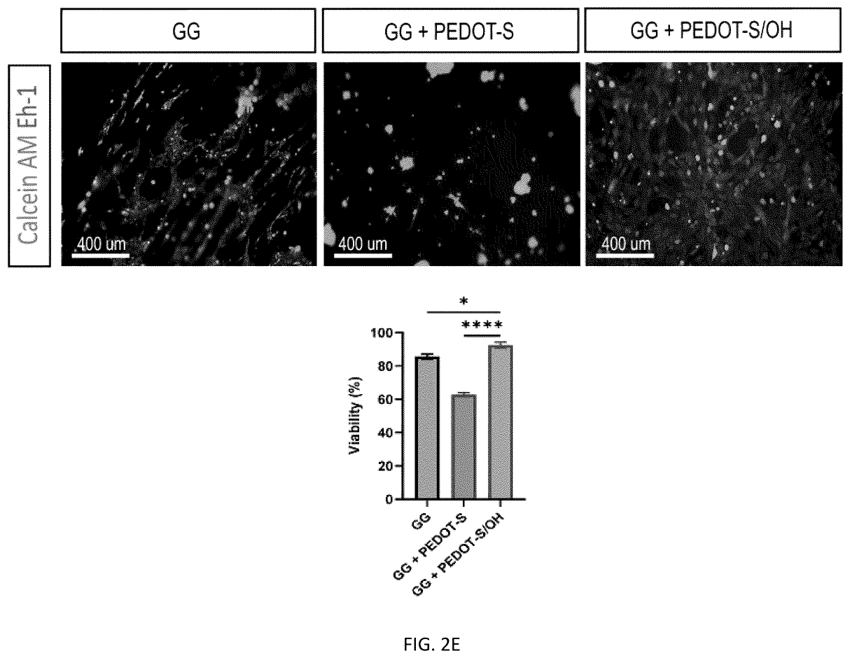

As shown in FIG. 2A, EDOT-S was combined with EDOT-OH to form a copolymer referred to herein as PEDOT-S/OH. The structure of PEDOT-S/OH is provided in FIG. 2A and is copied below.

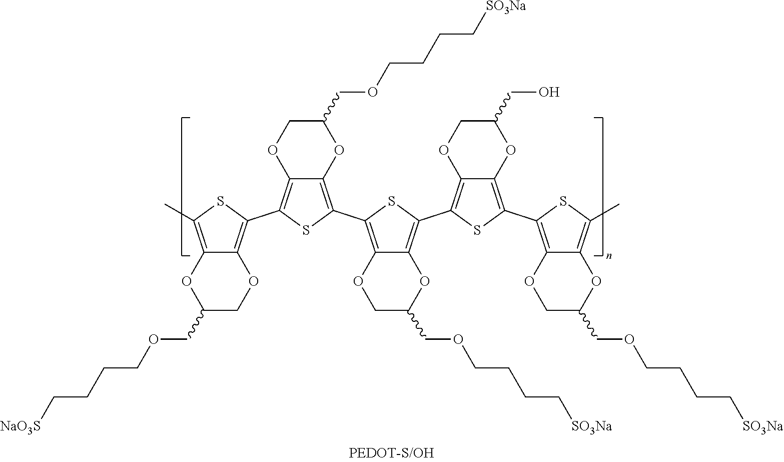

In some embodiments, the copolymer comprises the at least one cationic EDOT monomer and the at least one heterocyclic monomer (e.g. EDOT-OH) at a ratio of 1:10 to 10:1. For example, in some embodiments the copolymer comprises the at least one cationic EDOT monomer and the at least one heterocyclic monomer (e.g. EDOT-OH) at a ratio of 1:10 to 10:1, 1:2 to 9:1, 1:1 to 8:1, 2:1 to 7:1, 3:1 to 6:1, or 4:1 to 5:1. In some embodiments, the copolymer comprises the at least one cationic EDOT monomer and the at least one heterocyclic monomer (e.g. EDOT-OH) at a ratio of 1:10, 2:10, 3:10, 4:10, 1:2, 6:10, 7:10, 8:10, 9:10, 1:1, 2:1, 3:1, 4:1, 5:1, 6:1, 7:1, 8:1, 9:1 or 10:1. In some embodiments, the copolymer comprises the at least one cationic EDOT monomer and the at least one heterocyclic monomer (e.g. EDOT-OH) at a ratio of 2:1 to 6:1. In some embodiments, the copolymer comprises the at least one cationic EDOT monomer and the at least one heterocyclic monomer (e.g. EDOT-OH) at a ratio of 4:1.

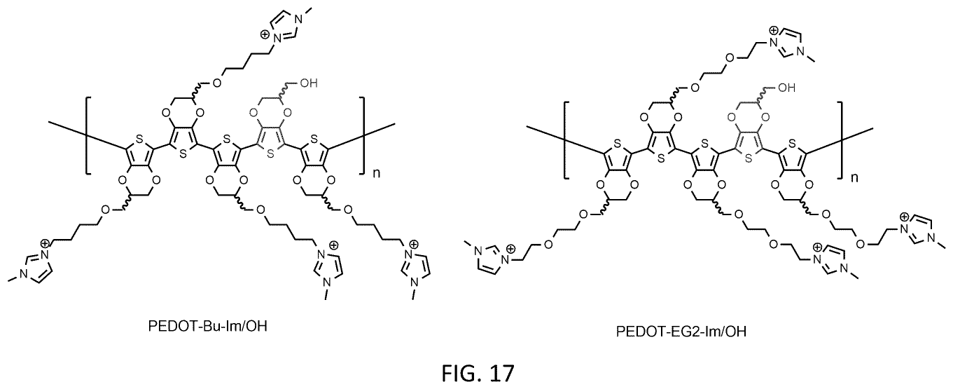

In some embodiments, the copolymer comprises four EDOT-imidazolium monomers and one EDOT-OH monomer. In some embodiments, the copolymer consists of four EDOT-imidazolium monomers and one EDOT-OH monomer. Exemplary structures of such a copolymer, referred to as PEDOT-imidazolium/OH, are provided in FIG. 17.

In some embodiments, a functional group is attached to another component by a linker. For example, in some embodiments heterocyclic monomer comprises a functional group which is attached to the heterocyclic monomer (e.g. the EDOT monomer) by a linker. In some embodiments, the functional group is attached to the linker covalently. In some embodiments, the functional group is attached to the linker and the linker is attached to EDOT by covalent interactions, the As another example, in some embodiments the hydroxyl group (e.g. the functional group) is attached to the dioxane ring in EDOT by a linker (thereby forming EDOT-OH). As another example, in some embodiments the methylimidazole is attached to the dioxane ring in EDOT by a linker. As another example, in some embodiments cationic functional group is attached to the EDOT monomer by a linker (thereby forming the cationic EDOT monomer). Exemplary linkers are shown in FIG. 12 and FIG. 16. However, any suitable linker may be used. In some embodiments, the linker comprises any combination of —CH₂—, —CH═CH—, —C≡C—, —O—, —NR′—, —BR′—, —S—, —C(O)—, —C(NR′)—, —S(O)—, —S(O)₂—, arylene, heteroarylene, cycloalkylene, and heterocyclylene moieties, wherein R′ is selected from hydrogen and C₁-C₆ alkyl, and wherein the arylene, heteroarylene, cycloalkylene, and heterocyclylene moieties are independently unsubstituted or substituted with 1, 2, or 3 substituents. In some embodiment, the linker is a peptide chain. In some embodiments, the linker comprises a peptide chain of length 1 to 20, 1 to 10, 1 to 5, or 1 to 3. For example, in some embodiments the linker comprises an alkylene chain where the number of amino acids is 1, 2, 3, 4, or 5.

In some embodiments, the linker comprises an alkylene chain. In other embodiments, the linker comprises an alkylene chain (e.g. comprising 2 to 20 CH₂ units) that is interrupted by, and/or terminated by (at either or both termini), at least one group selected from —O—, —S—, —N(R′)—, —CH═CH—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(CH₃)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C3-C₁₂ cycloalkylene, 3- to 12-membered heterocyclylene, 5- to 10-membered arylene, 5- to 12-membered heteroarylene, or any combination thereof, wherein each R′ is independently selected from hydrogen and C₁-C₆ alkyl, and wherein the interrupting and terminating groups may be the same or different.

In some embodiments, the linker comprises an alkylene chain:

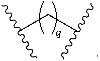

In some embodiments, the linker comprises an alkylene chain wherein q is 1 to 20, 1 to 10, 1 to 5, or 1 to 3. For example, in some embodiments the linker comprises an alkylene chain where q is 1, 2, 3, 4, or 5. For example, in some embodiments the linker comprises an alkylene chain that is terminated by an —O—, as shown in FIG. 12 and FIG. 16.

In some embodiments, the linker comprises one or more alkylene glycol repeat units, such as ethylene glycol or propylene glycol repeat units. For example, in some embodiments, the linker comprises a poly- or oligo-ethylene glycol chain:

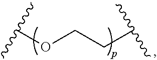

wherein p is 1 to 20, 1 to 10, 1 to 5, or 1 to 3. For example, in some embodiments, the linker comprises a poly or oligoethylene glycol chain

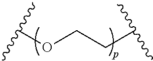

wherein p is 1, 2, 3, 4, or 5.

In some embodiments, provided herein are compositions comprising a copolymer described herein (e.g. PEDOT-S/OH). In some embodiments, provided here in is a composition comprising a copolymer described herein and a hydrogel material. A “hydrogel” refers to a soft crosslinked material capable of expanding in aqueous solvents and retaining water. Such a composition is also referred to herein as a “scaffold” and provides a suitable medium for cell growth and for promoting interaction of the cells within the scaffold with neighboring cells and tissue. In some embodiments, the composition comprises a linker that covalently links the copolymer to the hydrogel material. Any suitable linker may be used. In some embodiments, the linker is divinyl sulfone (DVS). In some embodiments, the copolymer is sufficiently retained within the hydrogel without a linker. For example, in some embodiments supramolecular interactions between the copolymer and the hydrogel material are sufficient to retain the copolymer within the hydrogel without a linker.

In some embodiments, the composition comprising a copolymer and a hydrogel material retains at least 90% of the copolymer within the hydrogel over the course of a 7 day period. Phrased alternatively, in some embodiments the leakage of the copolymer out of the hydrogel is less than 10% over the course of 7 days. In some embodiments, the leakage is less than 10%, less than 5%, less than 2%, or less than 1% over the course of 7 days. In some embodiments, the composition is conductive and also maintains high retention of the copolymer within the hydrogel.

In some embodiments, the hydrogel material comprises a polysaccharide. Exemplary polysaccharides suitable for use in hydrogels include, but are not limited to, alginate, hyaluronan, dextran, cellulose, carrageenan, pectin, chitosan, gellan, and the like. In some embodiments, the hydrogel material comprises gellan gum. In some embodiment, the gellan gum is high acyl. In some embodiment, the gellan gum is low acyl. In some embodiment, the hydrogel material gellan gum is low acyl. In some embodiment, the gellan gum is a mixture of high acyl and low acyl in different ratios. In some embodiments, a hydrogel is formed from 0-100% (mass %) low acyl gellan gum and 0-100% (mass %) high acyl gellan gum.

In some embodiments, the hydrogel material comprises a plurality of bioactive peptide amphiphiles. In some embodiments, the hydrogel material comprises gellan gum and a plurality of bioactive PAs. In some embodiments, the hydrogel material comprises a plurality of bioactive peptide amphiphiles, each bioactive peptide amphiphile comprising a hydrophobic tail, a structural peptide segment, a charged peptide segment, and a bioactive peptide. In some embodiments, the bioactive peptide is present in the extracellular matrix. The compositions described herein provide a physical, chemical, and electrical mimetic environment of the extracellular matrix, and thus find use in cell culture methods, methods of promoting cell growth and maturation, and/or methods of treating central nervous system injury.

In some embodiments, peptide amphiphiles comprise a bioactive moiety (e.g., IKVAV peptide). In particular embodiments, a bioactive moiety is the most C-terminal or N-terminal segment of the PA. In some embodiments, the bioactive moiety is attached to the end of the charged segment. In some embodiments, the bioactive moiety is exposed on the surface of an assembled PA structure (e.g., nanofiber). A bioactive moiety is typically a peptide, but is not limited thereto.

In some embodiments, the bioactive moiety is a peptide identified in the extracellular matrix (ECM). For example, the bioactive moiety may be a peptide sequence found in collagens, elastins, fibronectins, or laminins. In some embodiments, the bioactive moiety is a peptide sequence found in laminins. For example, the bioactive moiety may be found in laminin-1, laminin-2, laminin-3, laminin-4, laminin-5, laminin-6, laminin-7, laminin-8, laminin-9, laminin-10, laminin-11, laminin-12, laminin-13, laminin-14, or laminin-15. In some embodiments, the bioactive moiety is a peptide sequence found in laminin-1. In particular embodiments, the bioactive moiety is IKVAV (SEQ ID NO: 1). In some embodiments, the bioactive moiety is a recombinant peptide. In some embodiments, a bioactive moiety is a peptide sequence that binds a peptide or polypeptide of interests, for example, an ECM protein. In some embodiments, the bioactive moiety is a glycan.

In some embodiments, the composition (e.g. scaffold) comprises about 0.1% to 5% copolymer and about 95% to about 99.9% hydrogel (w/w). In some embodiments, the composition (e.g. scaffold) is generated by 3-D printing.

In some embodiments, the peptide amphiphile molecules are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, some embodiments described herein encompass peptide amphiphiles having a C-terminal moiety that may be selected from the group consisting of —H, —OH, —COOH, —CONH2, and —NH2.

In some embodiments, peptide amphiphiles comprise a hydrophobic segment (i.e. a hydrophobic tail) linked to a peptide. In some embodiments, the peptide comprises a structural peptide segment. In some embodiments, the structural peptide segment is a hydrogen-bond-forming segment, or beta-sheet-forming segment. In some embodiments, the structural peptide segment has the propensity to form random coil structures (e.g. a total propensity for forming (3-sheet conformations of 4 or less). In some embodiments, the peptide comprises a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more bioactive groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) to bury the lipophilic segment in their core and display the bioactive peptide on the surface. In some embodiments, the structural peptide undergoes intermolecular hydrogen bonding to form beta sheets that orient parallel to the long axis of the micelle. In some embodiments, the structural peptide displays weak intermolecular hydrogen bonding, resulting in a less rigid beta-sheet conformation within the nanofibers.

In some embodiments, PAs comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segment may comprise natural amino acids, modified amino acids, unnatural amino acids, amino acid analogs, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises E₂₋₄. For example, in some embodiments an acidic peptide segment comprises EE. In some embodiments, an acidic peptide segment comprises EEE. In other embodiments, an acidic peptide segment comprises EEEE (SEQ ID NO: 3).

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K.

In some embodiments, peptide amphiphiles comprises a structural peptide segment. In some embodiments, the structural peptide segment is a beta-sheet-forming segment. In some embodiments, the structural peptide segment displays weak hydrogen bonding and has the propensity to form random coil structures rather than rigid beta-sheet conformations. In some embodiments, the structural peptide segment comprises A₂G₂(SEQ ID NO: 2). In some embodiments, the structural peptide segment is rich in one or more of H, I, L, F, V, G, and A residues. In some embodiments, the structural peptide segment comprises an alanine- and valine-rich peptide segment (e.g., VVAA (SEQ ID NO: 4), VVVAAA (SEQ ID NO: 5), AAVV (SEQ ID NO: 6), AAAVVV (SEQ ID NO: 7), or other combinations of V and A residues, etc.). In some embodiments, the structural peptide segment comprises 4 or more consecutive A and/or V residues, or conservative or semi-conservative substitutions thereto. In some embodiments, the structural peptide segment comprises V₂A₂. In some embodiments, the structural peptide segment comprises an alanine and glycine-rich peptide segment (e.g. AAGG (SEQ ID NO: 2), AAAGGG (SEQ ID NO: 8), or other combinations of A and G residues, etc.).

In some embodiments, the structural peptide segment comprises 4 or more consecutive non-polar aliphatic residues (e.g., alanine (A), valine (V), leucine (L), isoleucine (I), methionine (M)). In some embodiments, the structural peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues. In some embodiments, the structural peptide segment comprises VEVA₂ (SEQ ID NO: 9).

In some embodiments, the structural peptide segment has a total propensity for forming β-sheet conformations of 4 or less (e.g. less than 4, less than 3.9, less than 3.8, less than 3.7, less than 3.6, less than 3.5, less than 3.4, less than 3.3, less than 3.2, less than 3.1, less than 3.0, less than 2.9. less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, or less than 1.)

In some embodiments, the structural peptide segment has a total propensity for forming β-sheet conformations of 4 or more.

The total propensity for forming β-sheet conformations may be calculated as the sum of the propensity for forming β-sheet conformations of each amino acid in the structural peptide segment. The propensity of each amino acid for forming β-sheet conformations and methods for calculating the same are described in, for example, Fujiwara, K., Toda, H. & Ikeguchi, M. Dependence of α-helical and β-sheet amino acid propensities on the overall protein fold type. BMC Struct Biol 12, 18 (2012), the entire contents of which are incorporated herein by reference. Exemplary values are shown in Table 1, below. For the purposes of calculating the total propensity for forming β-sheet conformations of the structural peptide segment, the value shown in the “total residues” column from table 1 for each amino acid is added together. For example, for an A2G2 structural peptide segment, the total propensity for forming β-sheet conformations is 0.75+0.75+0.67+0.67=2.84. The structural peptide segment may comprise any suitable number and combination of amino acids to achieve a total propensity for forming β-sheet conformations of 4 or less.

TABLE 1
Amino acid Propensities for β-sheet conformations

| Amino Acid | Exposed Residues | Buried Residues | Total Residues |
|---|---|---|---|
| V | 2.31 | 1.57 | 2.00 |
| I | 2.02 | 1.39 | 1.79 |
| L | 1.18 | 0.93 | 1.15 |
| M | 1.01 | 0.84 | 1.01 |
| P | 0.49 | 0.42 | 0.40 |
| A | 0.48 | 0.72 | 0.75 |
| C | 1.24 | 1.07 | 1.36 |
| F | 1.50 | 1.10 | 1.4 |
| Y | 1.71 | 1.12 | 1.37 |
| W | 1.90 | 0.91 | 1.23 |
| Q | 0.96 | 0.82 | 0.72 |
| S | 0.86 | 0.85 | .081 |
| T | 1.58 | 1.08 | 1.21 |
| N | 0.71 | 0.76 | 0.63 |
| H | 1.15 | 0.98 | 0.99 |
| D | 0.61 | 0.76 | 0.55 |
| K | 1.14 | 0.98 | 0.76 |
| E | 0.89 | 0.86 | 0.65 |
| R | 1.27 | 0.82 | 0.85 |
| G | 0/41 | 0.81 | 0.67 |

In some embodiments, a structural peptide segment having a total propensity for forming β-sheet conformations of 4 or less indicates that the amino acids within the structural peptide segment have weaker interactions with neighboring molecules. For example, the structural peptide segment may display weak hydrogen-bonding abilities. Accordingly, such structural peptide segments and the peptide amphiphiles comprising the same may create more dynamic nanofiber structures. For example, an A₂G₂(SEQ ID NO: 2) structural peptide segment may display random coil structures rather than rigid beta-sheet conformations.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group. In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH2(O(CH₂)₂)₂NH, CH2(O(CH₂)₂)₂NHCO(CH₂)₂CCH, etc.). In some embodiments, a spacer or linker further comprises additional bioactive groups, substituents, branches, etc. In some embodiments, the linker segment is a single glycine (G) residue.

Suitable peptide amphiphiles for use in the materials herein, as well as methods of preparation of PAs and related materials, amino acid sequences for use in PAs, and materials that find use with PAs, are described in the following patents: U.S. Pat. Nos. 9,044,514; 9,040,626; 9,011,914; 8,772,228; 8,748,569 U.S. Pat. Nos. 8,580,923; 8,546,338; 8,512,693; 8,450,271; 8,236,800; 8,138,140; 8,124,583; 8,114,835; 8,114,834; 8,080,262; 8,076,295; 8,063,014; 7,851,445; 7,838,491; 7,745,708; 7,683,025; 7,554,021; 7,544,661; 7,534,761; 7,491,690; 7,452,679; 7,371,719; 7,030,167; all of which are herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural peptide segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural peptide segment (e.g., AAGG (SEQ ID NO: 2)); and (c) a charged segment (e.g., comprising EE, EEE, EEEE (SEQ ID NO: 3), etc.). In some embodiments, any PAs within the scope described herein, comprising the components described herein, or within the skill of one in the field, may find use herein.

In some embodiments, a peptide amphiphile comprises (e.g., from C-terminus to N-terminus or from N-terminus to C-terminus): bioactive peptide (e.g., IKVAV (SEQ ID NO: 1) peptide)—charged segment (e.g., comprising EE, EEE, EEEE (SEQ ID NO: 3) etc.)—structural peptide segment (e.g., comprising A₂G₂(SEQ ID NO: 2) etc.)—hydrophobic tail (e.g., comprising an alkyl chain of 8-24 carbons).

In some embodiments, a peptide amphiphile comprises (e.g., from C-terminus to N-terminus or from N-terminus to C-terminus): bioactive peptide (e.g., IKVAV (SEQ ID NO:L 1) peptide)—flexible linker (e.g. comprising G, etc.)—charged segment (e.g., comprising EE, EEE, EEEE (SEQ ID NO: 3) etc.)—structural peptide segment (e.g., comprising A₂G₂(SEQ ID NO: 2)—hydrophobic tail (e.g., comprising an alkyl chain of 8-24 carbons).

In some embodiments, a PA further comprises an attachment segment or residue (e.g., K) for attachment of the hydrophobic tail to the peptide potion of the PA. In some embodiments, the hydrophobic tail is attached to a lysine side chain.

In some embodiments, provided herein are nanofibers and nanostructures assembled from the peptide amphiphiles described herein. In some embodiments, a nanofiber is prepared by the self-assembly of the PAs described herein. In some embodiments, a nanofiber comprises or consists of PAs displaying an IKVAV (SEQ ID NO: 1) peptide. In some embodiments, the IKVAV (SEQ ID NO: 1) peptides are displayed on the surface of the nanofiber. In some embodiments, in addition to PAs displaying IKVAV (SEQ ID NO: 1) peptides, filler PAs are included in the nanofibers.

In some embodiments, the composition (i.e. scaffold) comprises a conductive copolymer, one or more bioactive PAs, and one of more filler PAs. In some embodiments, filler PAs are peptide amphiphiles, as described herein (e.g., structural peptide segment, charged segment, hydrophobic segment, etc.), but lacking a bioactive moiety. In some embodiments, filler peptides are basic or acidic peptides lacking a bioactive moiety. In some embodiments, the filler PAs and IKVAV PAs self-assemble into a nanofiber comprising both types of PAs. In some embodiments, nanostructures (e.g., nanofibers) assembled from the peptide amphiphiles described herein are provided.

In some embodiments, filler peptides (e.g., basic peptide, acidic peptides, etc.) impart mechanical characteristics to a material comprising the PA nanofibers described herein. In some embodiments, a nanofiber assembled from 0-75% (mass %) bioactive IKVAV PA and 25-100% (mass %) basic filler PA becomes a gel at basic pH conditions (e.g., pH 8.5-11). In some embodiments, a nanofiber assembled from 75-100% (mass %) bioactive IKVAV PA and 0-25% (mass %) basic filler PA is a liquid at basic pH conditions (e.g., pH 8.5-11). In some embodiments, a nanofiber assembled from 0-20% (mass %) bioactive IKVAV PA and 80-100% (mass %) acidic filler PA becomes a gel at acidic pH conditions (e.g., pH 1-5). In some embodiments, a nanofiber assembled from 20-80% (mass %) bioactive IKVAV PA and 20-80% (mass %) acidic filler PA becomes a gel at neutral pH conditions (e.g., pH 5-8.5). In some embodiments, a nanofiber assembled from 80-100% (mass %) bioactive IKVAV PA and 0-20% (mass %) acidic filler PA is a liquid at acidic pH conditions (e.g., pH 1-5).

In some embodiments, nanostructures are assembled from (1) PAs bearing a bioactive moiety (e.g., IKVAV peptide) and (2) filler PAs (e.g., acidic or basic PAs not-labeled or not displaying a bioactive moiety, etc.). In some embodiments, nanostructures (e.g., nanofibers) comprise 95%, 90%, 85%, 80%, 75%, 70%, 65%, 60%, 55%, 50% 45%, 40%, 35%, 30%, 25%, 20%, 15%, 10%, 5%, 4%, 3%, 2%, or 1% (or any ranges there between) VEGF PAs. In some embodiments, nanostructures (e.g., nanofibers) comprise 95%, 90%, 85%, 80%, 75%, 70%, 65%, 60%, 55%, 50% 45%, 40%, 35%, 30%, 25%, 20%, 15%, 10%, 5%, 4%, 3%, 2%, or 1% (or any ranges there between) acidic filler PAs. In some embodiments, nanostructures (e.g., nanofibers) comprise 95%, 90%, 85%, 80%, 75%, 70%, 65%, 60%, 55%, 50% 45%, 40%, 35%, 30%, 25%, 20%, 15%, 10%, 5%, 4%, 3%, 2%, or 1% (or any ranges there between) basic filler PAs. In some embodiments, the ratio of IKVAV PA to acidic and/or basic PAs in a nanofiber determines the mechanical characteristics (e.g., liquid or gel) of the nanofiber material and under what conditions the material will adopt various characteristics (e.g., gelling upon exposure to physiologic conditions, liquifying upon exposure to physiologic conditions, etc.). In some embodiments, the nanostructures are assembled such that the nanostructures provide a hydrogel material suitable for retention of the copolymer within the hydrogel.

Peptide amphiphile (PA) nanofiber solutions may comprise any suitable combination of PAs. In some embodiments, at least 0.05 mg/mL (e.g., 0.10 mg/ml, 0.15 mg/ml, 0.20 mg/ml, 0.25 mg/ml, 0.30 mg/ml, 0.35 mg/ml, 0.40 mg/ml, 0.45 mg/ml, 0.50 mg/ml, 0.60 mg/ml, 0.70 mg/ml, 0.80 mg/ml, 0.90 mg/ml, 1.0 mg/ml, or more, or ranges therebetween), of the solution is a filler PA (e.g., without a bioactive moiety). In some embodiments, at least 0.25 mg/mL of the solution is a filler PA. In some embodiments, a filler PA is a non-bioactive PA molecule having highly charged glutamic acid residues on the terminal end of the molecule (e.g., surface-displayed end). These negatively charged PAs allow for the gelation to take place between nanofibers via ionic crosslinks. In some embodiments, a filler PA is a non-bioactive PA molecule having highly charged lysine residues on the terminal end of the molecule (e.g., surface-displayed end). These positively charged PAs allow for the gelation to take place under basic conditions. The filler PAs provide the ability to incorporate other bio-active PAs molecules into the nanofiber matrix while still ensuring the ability of the nanofibers solution to gel. In some embodiments, the solutions are annealed for increased viscosity and stronger gel mechanics. These filler PAs have sequences are described in, for example, U.S. Pat. No. 8,772,228 (e.g., C₁₆-VVVAAAEEE (SEQ ID NO: 10)), which is herein incorporated by reference in its entirety.

In some embodiments, the PA nanofiber described herein exhibit a small cross-sectional diameter (e.g., <25 nm, <20 nm, <15 nm, about 10 nm, etc.). In some embodiments, the small cross-section of the nanofibers (˜10 nm diameter) allows the fibers to permeate the brain parenchyma.

In some embodiments, the compositions described herein find use in cell culture methods. The compositions (e.g. compositions comprising a conductive copolymer described herein and a hydrogel material, such as a hydrogel material comprising a bioactive PA) described herein are capable of supporting growth and differentiation of a cell. In some embodiments, provided herein are methods comprising contacting a cell with a composition described herein. In some embodiments, the composition (i.e. scaffold) is used as a coating for any desired cell culture tool (tissue culture plate, petri dish, glass slide, etc.). In some embodiments, the compositions (i.e. scaffolds) provided herein provide electrically conductive materials which propagate signals between cells (e.g. neurons) cultured on the scaffold and neighboring cells/tissues, which furthers their communication and regeneration. In addition, the scaffolds may comprise a bioactive peptide amphiphile which promotes cell adhesion, neurite outgrowth, and/or other components of cell growth, maturation, and signaling. In some embodiments, cells (e.g. neurons) cultured on the compositions disclosed herein may demonstrate improved characteristics compared to cells cultured in the absence of the disclosed compositions. Surprisingly, inclusion of PEDOT-S/OH in a bioactive hydrogel is shown herein to provide an antioxidant effect. This antioxidant effect promotes cell maturation. For example, cells may demonstrate improved differentiation, improved maturation, improved electrical/cell signaling properties (e.g. action potentials, long term potentiation, long term depression), and/or improved long term viability compared to cells cultured in the absence of the disclosed compositions.

In some embodiments, the compositions provided herein are used in methods of treating disease. For example, the compositions described herein may be used for methods of treatment or prevention of nervous system injury in a subject. For example, the compositions may be used herein to promote the growth, maturation, differentiation, viability, etc. of neurons following a nervous system injury or affliction with a disease that causes damage or death to neurons in the subject. In some embodiments, provided herein are systems comprising a composition (e.g. a scaffold) described herein and a cell cultured on the scaffold. In some embodiments, such compositions (e.g. scaffolds) are provided to a subject having a nervous system injury or disease. In some embodiments, the scaffolds promote the health, growth, differentiation, viability, etc. of a cell cultured on the scaffold and thus find use in methods of cell replacement therapy to treat a nervous system injury or disease for which cell replacement therapy is beneficial. The compositions may improve outcomes following cell replacement therapy, such as by improving the overall health, maturity, viability, or activity of the transplanted cells in the subject.

In some embodiments, the systems and compositions described herein may be used in methods for treatment of prevention of injury to the central nervous system (CNS), including the brain and the spinal cord, or the peripheral nervous system (PNS), including the nerves and ganglia outside of the brain and spinal cord. In some embodiments, the systems and compositions herein may be used for treatment or prevention of injury to the CNS or PNS in a subject. In some embodiments, the injury is a spinal cord injury. The spinal cord injury may be cervical, lumbar, thoracic, sacral, or any combination thereof.

The injury may be a traumatic injury. A traumatic injury refers to an injury caused by trauma, for example trauma such as that caused by an automobile accident, a fall, violence, sports injury, surgical injury, and the like.) For example, the systems and compositions described herein may be used for the treatment of traumatic spinal cord injury. As another example, the systems and compositions described herein may be used for the treatment of traumatic brain injury (TBI). Alternatively, the injury may be a non-traumatic injury. For example, the injury may be a non-traumatic injury to the CNS (e.g., the brain and/or the spinal cord) or the PNS caused by, for example, cancer, multiple sclerosis, inflammation, arthritis, spinal stenosis, tumors, blood loss, stroke, and the like.

In some embodiments, the system or composition described herein is provided to a subject suspected of having a traumatic spinal cord injury. For example, the system or composition may be provided to the subject exhibiting one or more symptoms including loss of sensation and/or loss of motor control in one or more areas of the body (e.g. hands, arms, legs, feet, etc.), low blood pressure, inability to regulate blood pressure, inability to regulate body temperature, inability to sweat below the area of injury, chronic pain, and/or swelling of the spinal cord. The system or composition may be provided to the subject to treat the injury. In some embodiments, treating the injury may prevent worsening of one or more symptoms associated with the injury. In some embodiments, treating the injury may reduce the severity of and/or eliminate one or more symptoms associated with the injury.

A composition or a system described herein may be provided to a subject at any suitable point following injury or at any suitable point during progression of the disease (e.g. traumatic spinal cord injury) to treat the injury or disease. For example, the composition or system may be provided to the subject within 24 hours of the injury (e.g. within 24 hours, within 12 hours, within 10 hours, within 9 hours, within 8 hours, within 7 hours, within 6 hours, within 5 hours, within 4 hours, within 3 hours, within 2 hours, or within 1 hour from injury. In some embodiments, the composition or system may be provided to the subject after a duration longer than 24 hours has passed following injury or diagnosis of injury.

The composition or system may be administered in any suitable amount, depending on factors including the age of the subject, weight of the subject, severity of the injury, and the like. The composition may be administered in combination with other suitable treatments for injury or preventative measures to prevent the severity of the injury from worsening.

In some embodiments, the compositions herein are formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the composition is provided directly to the site of the injury. For example, in some embodiments the composition is provided (e.g. injected) directly to the site of the injury (e.g. directly to the spinal cord) and comes into contact with injured cells (e.g. injured neural cells). In some embodiments, the composition promotes signaling between neural cells proximal to the site of the injury and promotes cell maturation and outgrowth proximal to the site of the injury, thus repairing the injured tissue. In some embodiments, the compositions are administered parenterally. In some embodiments, parenteral administration is by intrathecal administration, intracerebroventricular administration, or intraparenchymal administration. The compositions herein can be administered as the sole active agent or in combination with other pharmaceutical agents such as other agents used in the treatment of nervous system injury in a subject.

### EXAMPLES

### Example 1

### Methods

### PEDOT-S/OH Synthesis and Purification

EDOT-S and EDOT-OH powders were first solubilized in Milli-Q water at 40 mM and 10 mM respectively. A polymerization solution of 2 eqv. sodium persulfate (NaPS) and 0.1 eqv. iron(III) chloride was added, and the solution was left to polymerize for 2 hours at room temperature. Following this, acetone was added to stop the polymerization. The tube was centrifuged, and the supernatant was removed. Acetone was added again, and the tube was spun for a total of 3× washes. The polymer was resuspended in Milli-Q and dialyzed overnight using a 10K MWCO cassette (ThermoFisher). PEDOT-S/OH was removed from the membrane, frozen in liquid nitrogen and lyophilized over several days. This resulted in a dark blue powder, which was stored at −20° C. until further use.

### GG+PEDOT Gel Preparation

PEDOT powders were solubilized in 150 mM NaCl+3 mM KCl at 0.1 wt % at RT while gellan gum (CP Kelco, MW 200-300 kDa, low acylated) was solubilized at 1.5 wt % at 80° C. Solutions were combined at 1:1 and left at 80° C. for 30 mins and then removed from the water bath to slow cool.

### PA+PEDOT Coatings Preparation

PEDOT and PA powders were solubilized in 150 mM NaCl+3 mM KCl at 0.1 wt % and 1 wt % respectively. Solutions were pH adjusted to ˜7.4 with 1 μM NaOH, and annealed at 80° C. for 30 mins and slow cooled overnight.

### Absorbance Spectroscopy

Gellan gum (GG) solution was prepared by solubilizing GG at 1.5 wt % in saline solution at 80° C., adding PEDOT powder at 0.1 wt %, and annealing for 30 minutes to a slow cool back to room temperature. Gellan gum+PEDOT hydrogels were crosslinked with 20 mM CaCl₂) solution and placed in saline solution for 1 week. Supernatant was removed and injected into a cuvette with 1 cm pathlength, with pure saline as a control. Absorbance spectra were collected using the Ocean Optics USB4000-UV-VIS, with data update rate=100 us, scans to average=100 and boxcar width=2.

### Conductivity Measurements

For electrical measurements, gels of GG+PEDOT were sandwiched between two ITO electrodes with a 2 mm spacer. For chronoamperometry, a constant 100 mV potential was applied and current was recorded over time. For electrochemical impedance spectroscopy, an alternating potential of 10 mV was applied over the frequency range from 100 kHz to 0.1 Hz. The curves were fit and normalized based on the electrode geometry.

### Rheology Measurements

Measurements were conducted on an Anton Paar MCR302 Rheometer with a 25 mm cone plate fixture. 150 μL of sample was placed on the sample stage, and the instrument was set to 23° C. The top fixture was lowered to the measuring position and a humidity collar was added to prevent evaporation. Oscillatory measurements with a constant strain of 0.1% were conducted over the course of 10 minutes and the storage and loss modulus were recorded. A strain sweep from 0.1 to 100% was also performed to measure the ductility of the gels.

### Elemental Analysis

PEDOT powder was sent to Midwest Microlab for CHNOS analysis. Samples were vacuum dried at 80° C. for 1 hour prior to measurement. Duplicate runs were averaged and compared to theoretical calculations of the relative element masses for various monomer ratios.

### NMR Sample Preparation

The five samples (gellan gum (GG), PEDOT-S (PS1), PEDOT-S/OH (PS2), GG+PS1, and GG+PS2) were prepared in MilliQ water with 20% D₂O and initially analyzed using solution-phase NMR. The samples were then subjected to ultracentrifugation for 4 hours at 15° C. and ˜600,000 g using a Sorvall MTX preparative ultracentrifuge with an S140-AT fixed angle rotor. In all cases, spinning led to the formation of pellets and supernatants. The supernatant was removed, and the pellet was packed in Kel-F inserts for 4 mm zirconia rotors for NMR analyses with magic angle spinning (MAS).

### NMR Data Acquisition and Processing

Solution-phase ¹H NMR spectra were recorded on a Bruker Avance III HD 500 MHz with a BBO Prodigy probe (sensitivity: 1H=1200, 13C=700) or a Bruker Neo 600 MHz system equipped with a QCI-F cryoprobe (sensitivity: 1H=5000, 13C=800). ¹³C NMR spectra were recorded on a Bruker Avance III HD 500 MHz with a BBO Prodigy probe (sensitivity: 1H=1200, 13C=700). Structural assignment was performed using 1H-1H-gCOSY, 1H-13C-gHSCQAD, 1H-1H-TOCSY. Multiplicities are quoted as singlet (s), doublet (d), triplet (t), double of doublets (dd), and multiplet (m). ¹H HRMAS NMR data were acquired on Bruker Avance Neo 600 MHz equipped with CMP-HRMAS from Bruker at 38° C. and MAS speed of 4.0 kHz. ¹³C CPMAS and rINEPT NMR spectra were acquired at 38° C. and an MAS speed of 13.3 kHz using Bruker Avance III 600 MHz equipped with a 3.2 mm Phoenix HFXY probe. The probe was configured to HX double-resonance mode for optimal ¹³C sensitivity. The chemical shifts are reported in parts per million (ppm) with tetramethylsilane (TMS), 3-(trimethylsilyl)propionic-2,2,3,3-d₄ acid, or sodium 2,2-dimethyl-2-silapentane-5-sulfonate (DSS) as internal standard. The spectra were processed in Topspin 4.1.4 from Bruker or Mnova 14.3.3.

### Aqueous Gel Permeation Chromatography (GPC)

Samples of PS1 and PS2 were prepared in basic water (pH˜10). GPC was conducted in the Northwestern Integrated Molecular Structure Education and Research Center (IMSERC) on an Agilent Aqueous GPC using PSS MCX column combination medium HR, composed of 1×PSS 5μ MCX 103 Å and 1×PSS 5μ MCX 105 Å with PSS 5μ MCX guard column.

### C2C2 Cell Culture

C2C12 cells were seeded onto gellan gum and PEDOT gels and cultured in DMEM (Gibco) media containing 10% FBS (Gibco)+1% penicillin/streptocycin (Sigma Aldrich). Full media change was performed 24 hours post seeding.

### E16 Primary Mouse Cortical Neuron Dissection and Culture

Cortical neurons were obtained from embryonic day 16 (E16) mouse brains as previously described. (Ortega, J. A.; Alcintara, S., Cereb Cortex 2010, 20 (9), 2132-44.) Briefly, a time pregnant CD1 mouse was sacrificed by anesthesia and further cervical dislocation. The embryos were extracted and dissected cortices were transferred into 5 mL of 0.25% Trypsin/EDTA solution with 250 μL of DNAse for 10 minutes. Cortices were then transferred into a 50 mL Falcon tube with 5 mL of media and 250 μL of DNAse and mechanically dissociated. Media consisted of neurobasal media (Gibco) containing penicillin/streptomycin (Sigma Aldrich), L-Glutamine (Gibco), sodium bicarbonate (Gibco), and NHS (Gibco). After 30 seconds, the supernatant was removed, placed in another Falcon tube, and centrifuged at 1000 rpm for 5 minutes. The supernatant was removed, and the cell pellet was resuspended in 2 mL of media. This solution was transferred into a cell culture petri-dish containing 8 mL of media for pre-plating. The dish was incubated for 40 minutes at 37° C. to allow for astrocytes to bind to the plate surface while neurons remained suspended in the solution. After pre-plating, the suspended neurons were filtered (100 m pore size) and transferred into a new 50 mL Falcon tube. The cells were centrifuged for 1000 rpm for 5 minutes, the supernatant was removed, and cells were resuspended in 2 mL of culture media. Following cell counting, cells were diluted with the appropriate amount of media and plated accordingly. After 24 hours, the entire media was replaced and then half changed twice weekly.

### Scanning Electron Microscopy

PA and PEDOT materials were prepared by solubilizing lyophilized PA powder at 1 wt % in a saline solution of 150 mM NaCl and 3 mM KCl prepared in Milli-Q water. PEDOT-S or PEDOT-S/OH was prepared by solubilizing the lyophilized powder at 0.1 wt % in the same saline solution and adding into the PA solution in a 1:1 ratio (Final concentrations are 1 wt % PA and 0.1 wt % PEDOT). The samples were bath sonicated and pH adjusted to ˜7.4 with the incorporation of NaOH. Samples were then annealed at 80° C. for 30 minutes and slow cooled at 1° C./minute to room temperature. 2D PA coatings were prepared on 12 mm glass coverslips by first adding a layer of PDL. PDL at 0.001 wt % solution was incubated on autoclaved glass coverslips for 2 hours at 37° C. After incubation, the solution was removed, the coverslips were washed 2× with autoclaved Milli-Q water and left to dry overnight. The PA coatings were then “painted” onto the PDL surface with a pipette and left to dry. Following hydration with 5 mM CaCl₂ solution and 2× washes with media, primary cortical neurons were cultured on the surface over time. Cells and PA glass coverslips were fixed in a 2% PFA+2.5% glutaraldehyde solution. An ethanol exchange was then performed prior to critical point drying (CPD). Samples were submerged for 15 minutes in each of the following ethanol solutions, in order: 30%, 40%, 50%, 60%, 70%, 80%, 85%, 90%, 95%, 100%. Afterword, the samples underwent CPD using a Tousimis samdri-795. Prior to SEM imaging, samples were coated with 16 nm of osmium using an SPF osmium coater. SEM imaging was performed using the Hitachi S-4800 SEM.

### Live/Dead Staining

Cortical neurons were cultured in well plates as previously described. After 48 hours, the media was removed, and wells were washed 3× with 1× HBSS. Calcein AM and ethidium homodimer 1 were added at 2 L/mL and 1 L/mL respectively in 1× HBSS. Plates were incubated for 20 minutes at 37° C. and washed 3× with HBSS again. Wells were then imaged using the IncuCyte microscope. Image quantification was done using ImageJ software.

### Immunocytochemistry

Neurons were fixed in 4% paraformaldehyde (Fisher Scientific, cat. no 043368.9M) for 20 min, after which they were washed with 1× PBS (Gibco, cat. no. 10010), blocking buffer prepared with 5% NHS (Gibco, cat. no. 16-050-122) and 0.1% Triton X-100 (Fisher Bioreagents, cat. no. BP151-500) in 1× PBS. Fresh blocking buffer was added for a 2 hour incubation. After this, the blocking buffer was replaced with the desired primary antibody solution (prepared in blocking buffer), which was incubated at 4° C. overnight. The next day, samples were washed with blocking buffer three times for 15 min each. 500 μL of the secondary antibody solution was added. Samples were incubated with a secondary antibody solution prepared in blocking buffer for 2 hours, protected from light. Following one wash in blocking buffer, the nuclear stain DAPI (1:1000, Thermofisher, D1306) was applied for 10 min. Samples were then washed three times with blocking buffer for 15 min each, followed by three washes with 1× PBS for 15 min each. Coverslips were mounted onto glass slides with Immuno-Mount solution (ThermoScientific, cat. no. 9990402) and stored at 4° C. until imaging. Primary antibodies: 1:2000 anti-MAP2 (Rb, Biolegend, 840601), 1:2000 anti-TUJ1 (Rb, Biolegend, 802001), 1:1000 anti-pCREB (Rb, CellSignaling, 9198), 1:1000 anti-CREB (Rb, CellSignaling, 9197). Secondary antibodies: 1:1000 Alexa Fluor 488 (Ms, Invitrogen, A-21202), 1:1000 Alexa Fluor 488 (Rb, Invitrogen, A11008), 1:1000 Alexa Fluor 555 (Rb, Invitrogen, A-31572), 1:1000 Alexa Fluor 647 (Ms, Invitrogen, A31571), 1:100 DAPI (Thermofisher, H1399).

### Western Blot

E16 primary cortical mouse neurons were cultured for 5 days on PDL, PA, and PA+PEDOT-S/OH coatings. Cells were seeded in a 6 well plate with 250,000 cells/well. Following culture, a solution of RIPA buffer with 1% protease and phosphatase inhibitor was added and cells were mechanically disrupted by a cell scraper. Samples were extracted and horn sonicated 3× at 10% for 10 seconds each time. Protein content was quantified using a bovine serum albumin (BSA) assay and measured using absorbance at 540 nm using a Cytation3 plate reader. Aliquots of 10 g protein for each sample were prepared prior to electrophoresis. Samples were run through 10-well 4-20% agarose gels at 120 V. Gels were then transferred to polyvinylidene difluoride (PVDF) membranes at 100 V for 1.5 hours. Ponceau solution was used to verify protein transfer to membranes.

Prior to antibody staining, membranes were blocked with 10% BSA in TBST buffer. Primary antibodies were added and incubated overnight at 4° C. The next day, the primary antibody was removed, and membranes were washed 3× with TBST buffer. Secondary antibodies were added in 10% BSA in TBST at room temperature for 1 hour. Membranes were washed 3× with TBST buffer and soaked in Radiance prior to imaging. Membranes were imaged using an Azure300 gel imager.

### Sholl Analysis

Morphology analysis was performed with Fiji software on confocal images of DIV 3 neurons immunolabeled with MAP-2 antibody. Simple Neurite Tracer (SNT) plugin was used to semi-automatically reconstruct images and adjust for brightness and contract. The Sholl analysis plugin was then used to analyze branching complexity of neurons through a series of concentric circles formed around the cell body. These calculate the number of branches crossing each circle to create a graphical representation of branching quantity as radial distance from the soma increases.

### Electrophysiology Measurements

Whole cell patch clamp was performed using 2-4 MΩ glass electrodes pulled from glass capillary tubes with a Flaming-Brown P-97. Electrodes were positioned using a Sutter Instrument MP-285 motorized micromanipulator. Measurements were performed at RT using the Multiclamp700B amplifier (Molecular Devices) and Winfluor software. Briefly, coverslips were perfused with a modified Ringer's solution containing (in mM): 111 NaCl, 3.09 KCl, 25.0 NaHCO₃, 1.10 KH₂PO₄, 1.26 MgSO₄, 2.52 CaCl₂), and 11.1 glucose. The solution was oxygenated with 95% 02 and 5% C02 and perfusion rates were 1.5-2.0 ml/min. Patch electrodes contained (in mM) 138 K-gluconate, 10 HEPES, 5ATP-Mg, 0.3 GTP-Li and Texas Red dextran (75 μM, 3000 MW, Invitrogen). In voltage-clamp mode, fast and slow capacitance transients, as well as whole-cell capacitance was compensated using the automatic capacitance compensation on the Multiclamp. In current clamp, neurons were subjected to depolarizing current ramps for testing the current level at firing onset, the current level at cessation of firing, and the frequency-current relationship. Hyperpolarizing current was used to hold neurons near ˜80 mV in between stimuli. Neurons selected were large (input resistance<1000 MΩ) and had a resting membrane potential −35 mV or less. The first action potential evoked by a depolarizing current ramp was used to measure all parameters, including the current at firing onset. Threshold voltage was defined as the voltage at which the slope exceeded 10 V/s. Action potential size was measured using overshoot (past 0 mV) minus threshold voltage. Duration of the action potential is measured at half of action potential height. Rates of rise and fall are defined as the peak and the trough of the first derivative of the action potential profile.

### RNA Sequencing and Gene Ontology

Cells were lysed using RPE lysis buffer and RNA extracted using a RNA MicroEasy kit (QIAGEN). RNA purity was determined using a NanoDrop Microvolume Spectrophotometer (ThermoFisher). Samples with A260/A280 ratios between 1.8-2.2 were considered acceptable for RNAseq analysis. Samples were sent to NUSeq core for RNA library analysis and Metascape gene ontology analysis.

### Reactive Oxygen Species (ROS) Studies

The presence of ROS was determined using CellROX Green Reagent (ThermoFisher). Briefly, CellROX was added to cells for a final concentration of 5 μM and incubated for 30 mins at 37° C. Wells were washed 3× with PBS and fluorescence intensity was read at 485/520 nm using Cyation3. For solution studies, CellROX was added to solutions of PEDOT or PA containing 10 μM H₂O₂ at various time points.

Human Induced Pluripotent Stem Cell (hiPSC) Culture

Induced pluripotent stem cells (iPSCs) were maintained on Matrigel-coated dishes supplemented with mTeSR1 (Stem Cell Technologies). Medium change was performed every day. For cell passaging or differentiation, cells were dissociated to single cells with 1 mM EDTA or Accumax (Innovative Cell Technologies), respectively. Matrigel lots were routinely tested to optimize stem cell maintenance. ROCK inhibitor (RI) was used to prevent cell death upon cell dissociation 2. The stem cell markers were routinely validated by immunostaining and alkaline phosphatase assay.

### Spinal Cord Organoid Culture

Spinal cord organoids (hSCOs) were produced from iPSCs. Briefly, the iPSCs were seeded at 10K cells per well in the low-cell-adhesion U-bottomed 96 wells (Sumilon PrimeSurface plate). A series of growth medium instructions was performed as follows: from day 0 to day 3, 10 μM RI, 20 ng/ml recombinant human basic FGF, 3 μM CHIR99021 and 10 M SB431542 were added. From day 3 to day 6, the tissues were then supplemented with 10 M SB431542, 500 nM Smoothened Agonist (SAG) and 100 nM retinoic acid (RA). From day 6 to day 15, half the medium was performed with 500 nM SAG and 100 nM RA. On day 15, the hSCOs were transferred to 6 well dish in spinal cord maturation medium containing N2B27 medium supplemented with 1 mM L-Glu, 0.1 mM 2-ME, 0.5 μM ascorbic acid, 10 ng/ml BDNF, 20 ng/ml GDNF and 100 nM RA. N2B27 consists of DMEM/F-12 (Gibco), neurobasal medium (Gibco) (1:1), 0.5% (vol/vol) N2 supplement (ThermoFisher) and 1% (vol/vol) B27 supplement without vitamin A (Invitrogen).

### 3D Printing

Gellan gum and PA+PS2 gels were heated to 60° C. for 30 min and loaded together into printing syringes (Nordson EFD). Scaffolds were printed into 24 well plates on a BioX Printer (CELLINK) using a 22 G conical tip, printing speed of 15 mm/sec, force 20 kPa. 3D models were created in AutoCAD software. Prints were washed with 20 mM CaCl₂) and kept in Neurobasal media (Gibco)+1% penicillin/streptomycin (Sigma Aldrich. Murine cortical neurons were seeded on the prints using the aforementioned methods and cultured for 1 week.

### Polarized Light Microscopy (POM)

Printed structures were printed using aforementioned methods onto glass coverslips and soaked in 20 mM CaCl₂) to crosslink the filaments. POM was performed using the EVOS M5000 Imaging System. with the slides placed between two perpendicular light polarizers. Images were collected with the filaments oriented at 0° and 450 to the polarizer using the same exposure time.

### Statistical Analysis

Data analysis was performed using GraphPad Prism (version 10.1.0). Comparisons among three or more groups were conducted using one-way ANOVA with a Tukey's multiple comparisons test. The statistical tests and parameters used for each experiment are depicted in the corresponding figure legends. For confocal data, error bars represent at least 20 images from two to three separate dissections. For Western blot experiments, error bars represent densitometry results calculated across three distinct blots from three separate dissections. For patch clamp experiments, error bars represent three distinct wells from two dissections. All error bars shown in the graphs represent the standard error mean unless otherwise indicated.

### Results

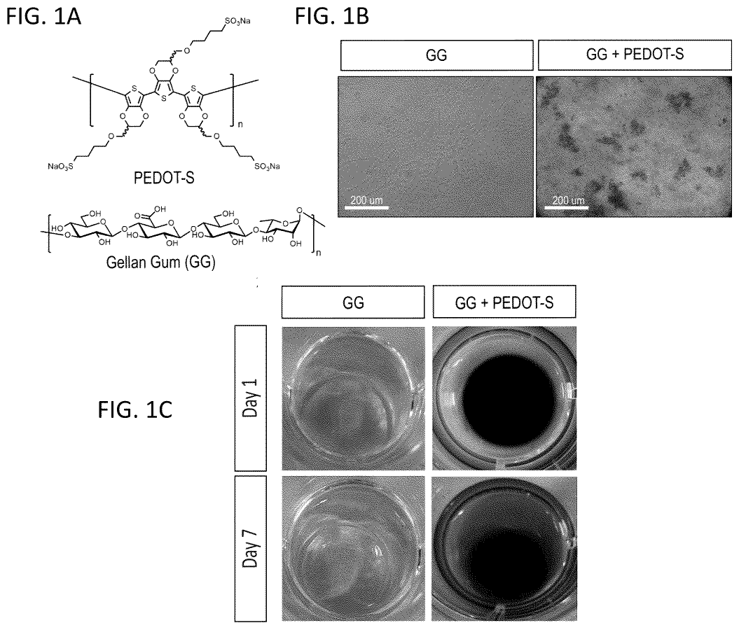

To test the biocompatibility of PEDOT-S polymer, PEDOT-S was combined with gellan gum (GG) which formed a conductive self-standing hydrogel (FIG. 1a). C2C12 murine myoblasts were cultured on these gels for 3 days. Cells cultured on the GG alone depicted a natural elongated morphology, while the incorporation of PEDOT-S (PS1) resulted in more rounded cells indicating apoptosis (FIG. 1b). The membranes of cells cultured on GG+PEDOT-S were stained dark blue, possibly because of the amphiphilic PEDOT-S intercalating into the membrane and causing cell lysis. A blueish hue in the cell media was also observed, indicative of the PEDOT-S leaking out of the GG hydrogel (FIG. 1c). Thus, a functionalized PEDOT-S that would maintain conductivity but prevent leakage from the hydrogel scaffold was developed.

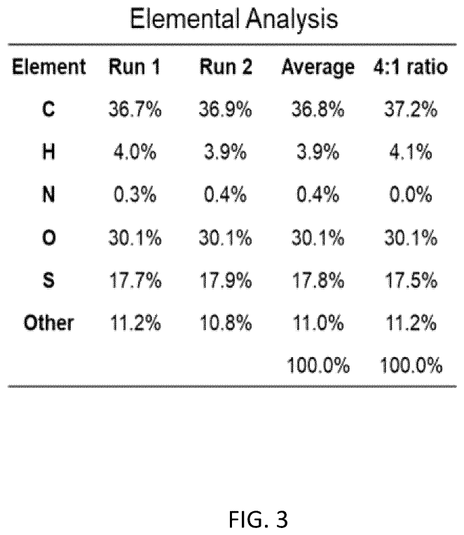

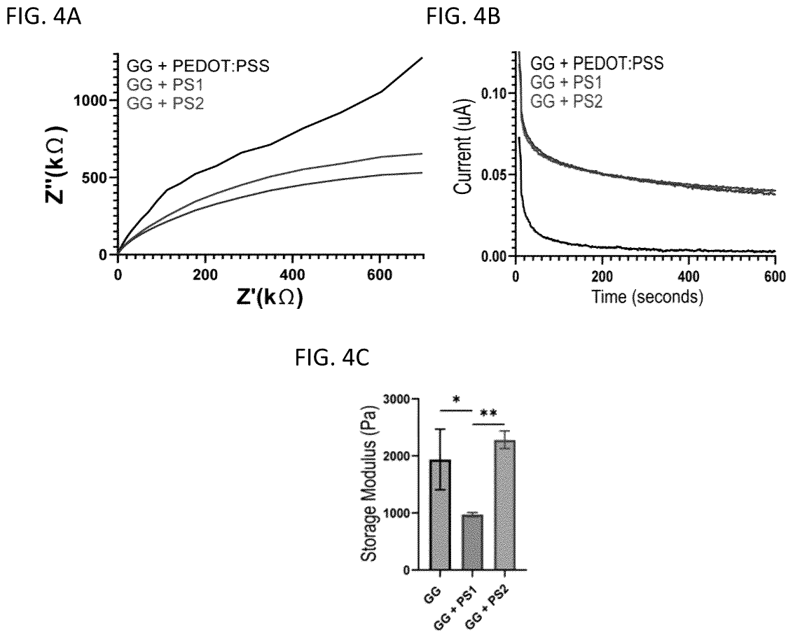

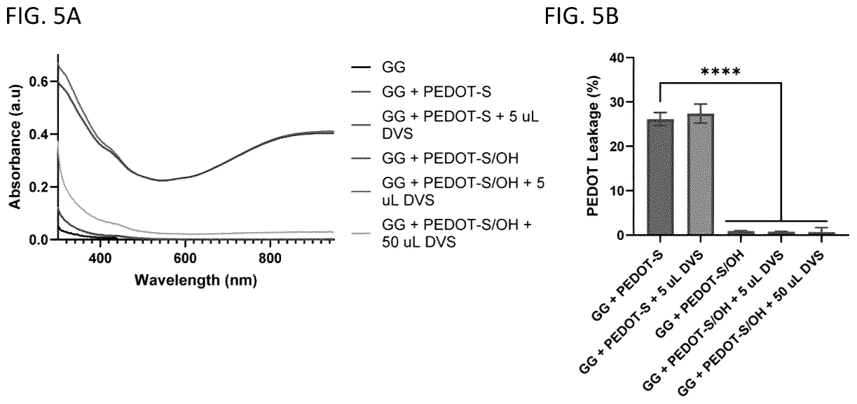

To address the PEDOT-S leakage issue, a copolymer consisting of EDOT-S and hydroxy-EDOT monomers was synthesized that could result in a conductive PEDOT-S/OH (PS2) with a handle for crosslinking through the —OH group (FIG. 2a). Using elemental analysis, the monomer ratio in the copolymer was found to be 4:1 of EDOT-S:EDOT-OH (FIG. 3). When mixed into GG hydrogels, PEDOT-S/OH (PS2) had similar conductivity to PEDOT-S and the same rheological properties as pure GG gel (FIG. 4). When a commercially available linker (divinyl sulfone, DVS) was used to covalently link the hydroxy-EDOT groups to hydroxyls on the GG backbone, a significant decrease in leakage of PEDOT-S/OH out of the gels when placed in saline solution was used (FIG. 5). Surprisingly, GG gels mixed with PEDOT-S/OH even without DVS resulted in minimal leakage (FIG. 2b, c, d). The enhanced retention of the copolymer in the GG matrix without a covalent crosslinker suggested that supramolecular interactions (e.g., hydrogen bonding between the EDOT-OH and GG moieties) were responsible for enhanced retention of the PEDOT-S/OH.

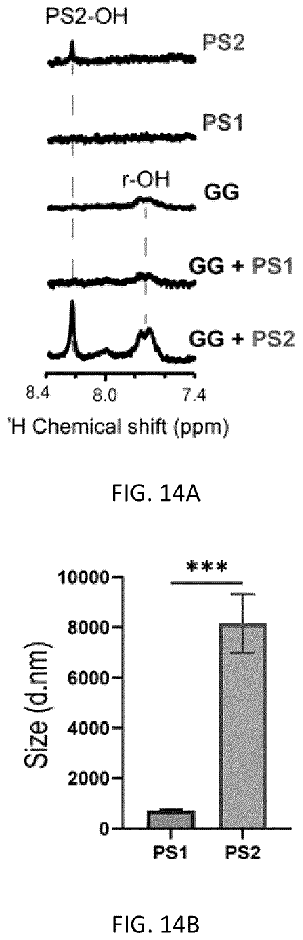

To probe these interactions and the enhanced retention of the copolymer in the GG hydrogel, high-resolution magic angle spinning (HRMAS) nuclear magnetic resonance (NMR) spectroscopy was performed. This technique is highly effective for observing ¹H in liquid and semi-solid states and well-established for studying diverse samples, including biomolecular and synthetic polymers. Hydroxyl hydrogens from GG and PS2 were identified in the 7.40-8.40 ppm region (FIG. 14a). The singlet at 8.23 ppm in PS2 was assigned to its hydroxyl group. Interestingly, no copolymer singlets were identified in PS1. This singlet shows enhanced line broadening in GG+PS2 (FWHM=24.4 Hz) relative to PS2 (FWHM=3.8 Hz) which may be attributed to intermolecular hydrogen bonding between GG and PS2 (GGbound) in GG+PS2. The broad signal at 7.71 ppm was assigned to the hydroxyl groups of GG, based on its presence in the GG spectrum. ¹H-¹³C HMQC and ¹H 2D NOESY was also conducted. It was determined that this binding likely involves conformational reorganization of GG chains to maximize noncovalent attractions to PS2. These NMR features support the hypothesis that EDOT-OH in PS2 stabilizes GG and prevents leakage.

In addition to the intermolecular interaction between GG and PS2 shown by NMR, dynamic light scattering measurements showed that PS2 forms particles that are about four times larger than those of PS1 (FIG. 14b). PS2 co-polymers are as less water soluble than PS1, which further explains its decrease in leakage in our system.

Having established the enhanced retention of PEDOT-S/OH in GG gels, it was then tested if this new formulation would improve the viability of C2C12 myoblasts cultured on these gels. Indeed, myoblasts cultured on the PEDOT/S—OH gels had significantly enhanced survival by live-dead staining and LDH measurements (FIG. 2e). As such, the copolymer PS2 was found to be stable and biocompatible.

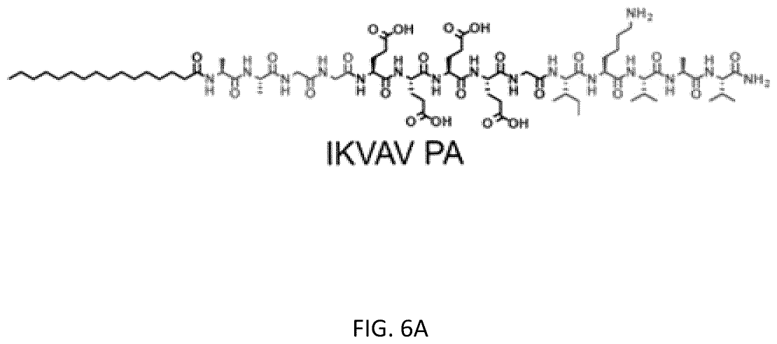

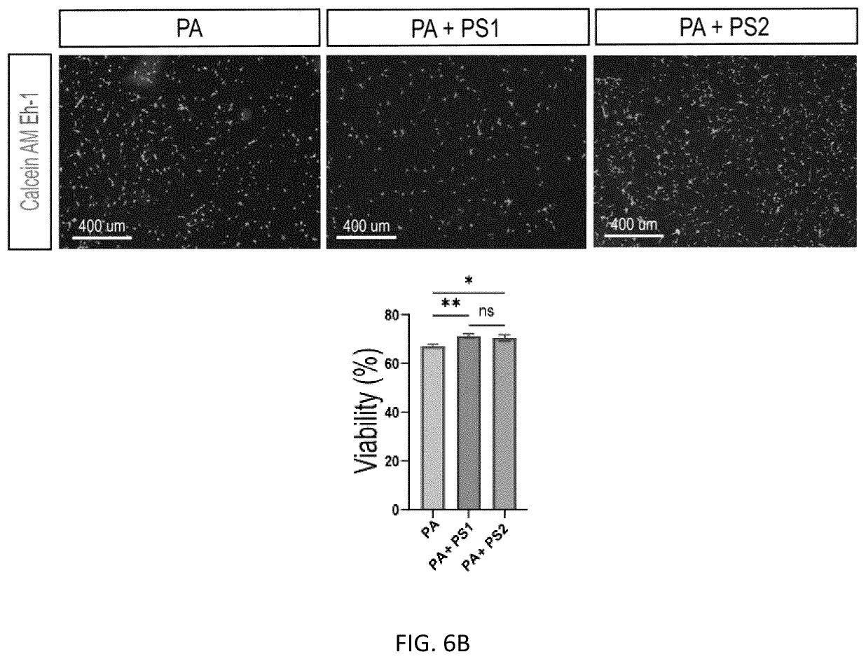

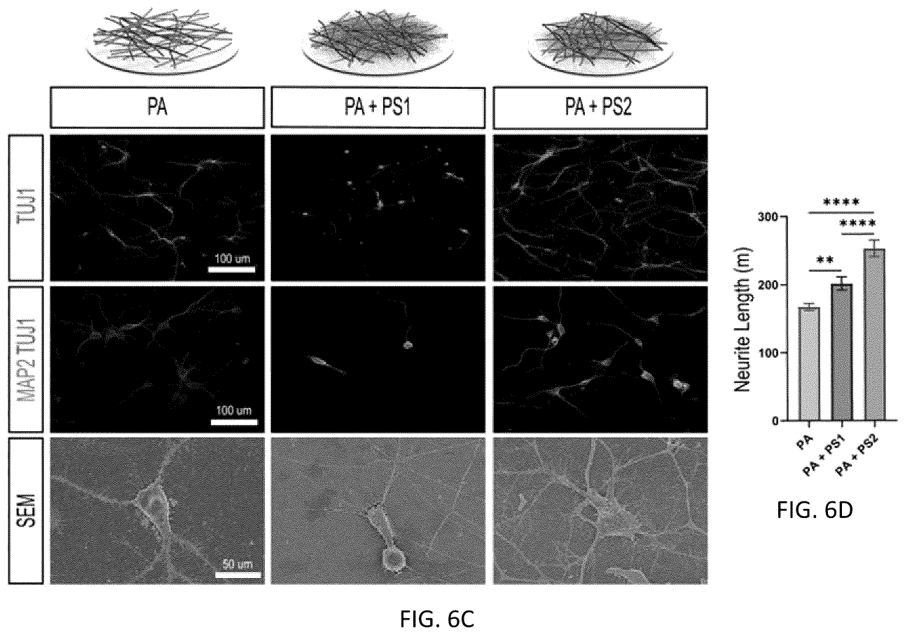

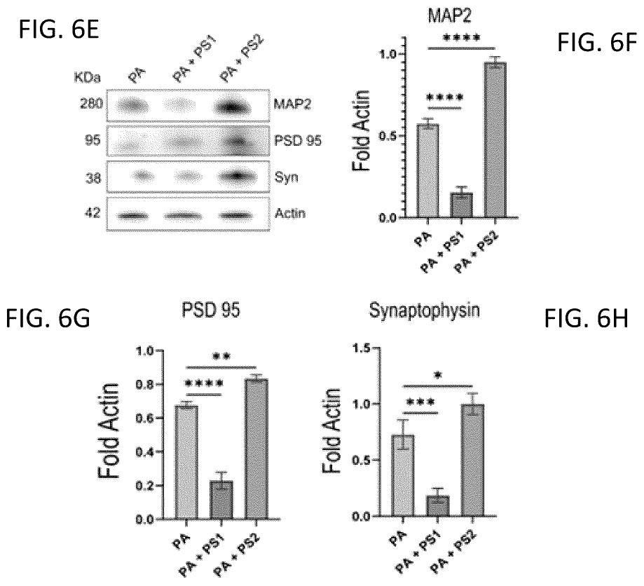

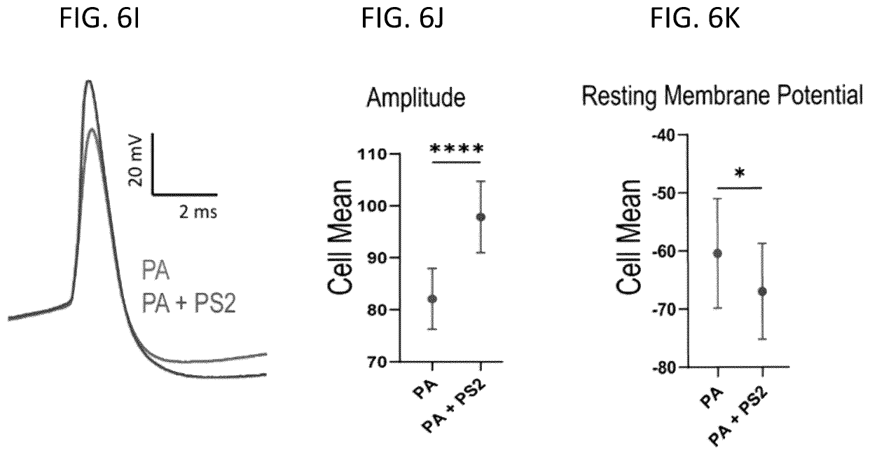

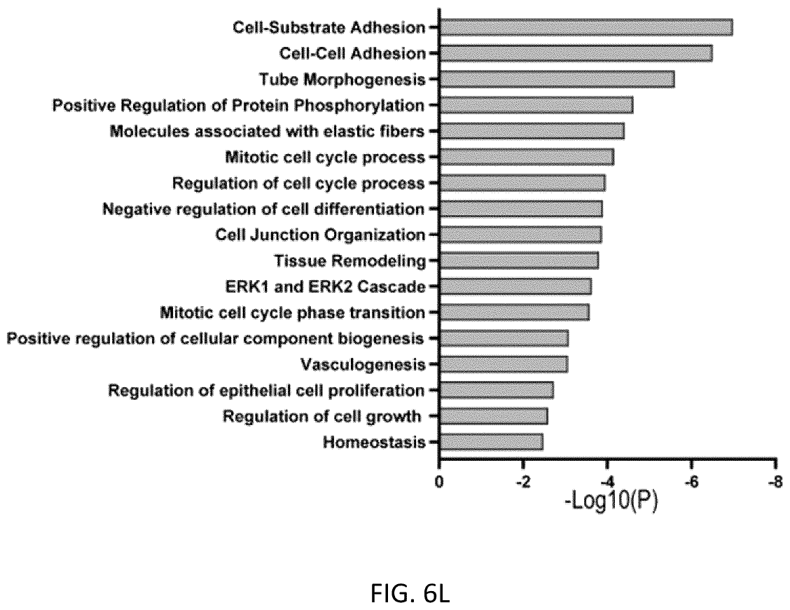

Having established the biocompatibility of the PEDOT-S/OH copolymer even without covalent crosslinking to a hydrogel scaffold, the conductive material was next combined with bioactive PA hydrogels. A PA molecule (C₁₆-A₂G₂E₄GIKVAV (PA) that displays a laminin mimetic pentapeptide (IKVAV) that has previously demonstrated neural stem cell maturation and differentiation in vitro, and enhanced functional recovery in a murine model of spinal cord injury, was used. PEDOT materials were added in solution with C₁₆-A₂G₂E₄GIKVAV (PA) and annealed together for 30 minutes at 80° C. E16 mouse primary cortical neurons were cultured on IKVAV PA with either PEDOT-S or PEDOT-S/OH to determine if the addition of the conducting polymer would improve neural maturation (FIG. 6a, b). Coatings of PA+PS2 showed lower cytotoxicity compared to PA alone after 2 days (FIG. 6c). After 5 days, the cells were stained with microtubular marker β-tubulin III (TUJ1), dendritic marker microtubule-associated protein 2 (MAP2), and nuclear stain 4′,6-diamidino-2-phenylindole (DAPI) (FIG. 6D). Cells on the coatings were also imaged using scanning electron microscopy (SEM), which showed enhanced secondary branching on PA+PS2. Morphological differences between conditions were further evaluated by quantifying neurite length in ImageJ. After 5 DIV, longer neurite lengths in cells cultured on the IKVAV coatings that incorporated the PEDOT materials were observed (FIG. 6E). However, due to the initial cytotoxicity of PEDOT-S, less cells were present in this condition. In comparison, in the PA+PEDOT-S/OH treatment, neurite length was further increased at all time points, which was complemented with a higher cell viability. Interestingly, in scanning electron microscopy (SEM) imaging of the PA+PEDOT-S/OH condition the presence of many secondary branches extending from primary neurite extensions (FIG. 6c) was observed. This indicates the formation of many filipodia/spines in the neurons, which represents increased cell attachment and synapse formation. Overall, these observations demonstrate that the addition of PEDOT-S/OH to IKVAV scaffold leads to a more mature neural network.

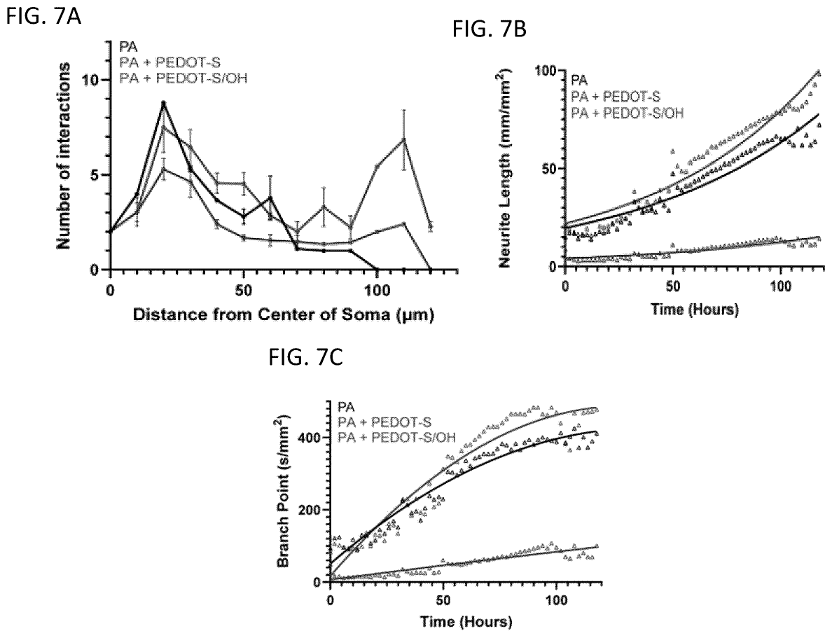

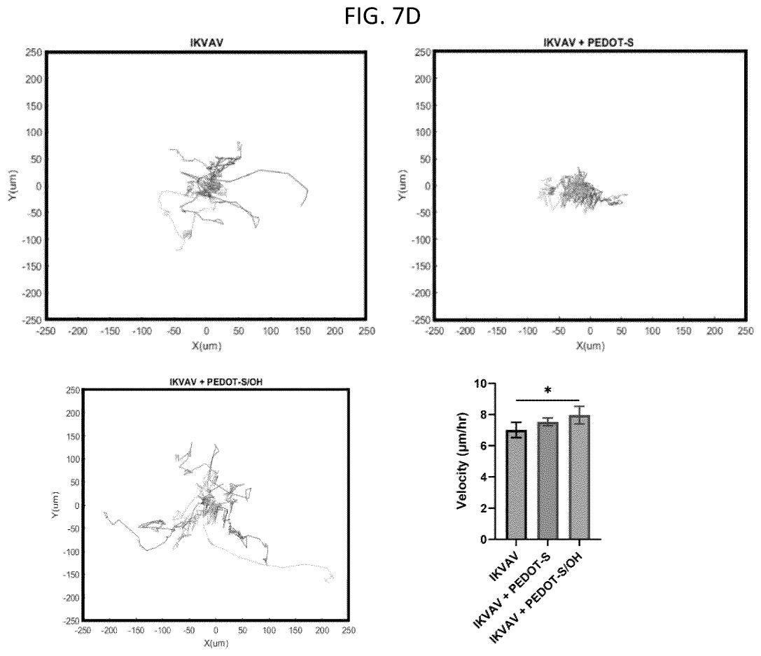

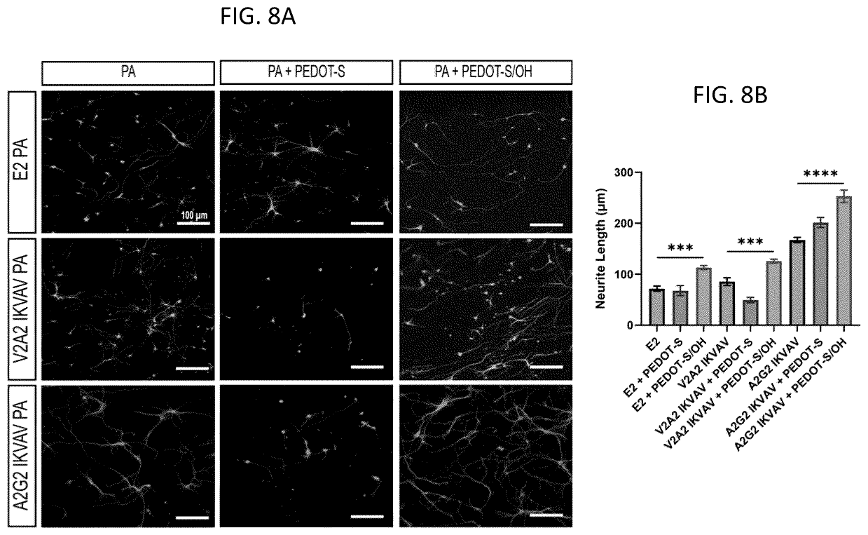

To complement these results, a Sholl analysis was performed on 3 DIV cortical neurons (FIG. 7a). PA+PEDOT-S/OH showed the largest amount of branching points and branching longer distances from the soma, indicating enhanced dendrite and spine formation. A time course analysis from DIV1-5 was also conducted to visualize and quantify neurite growth. These results corroborated the immunostaining imaging, where faster neurite outgrowth and more branching in the PA+PEDOT-S/OH was observed (FIG. 7b, c). A drastic increase in soma mobility of neurons cultured on the PA+PEDOT-S/OH coatings was also seen (FIG. 7d). To determine whether the increased neurite length was due to the PA epitope or the conducting polymer, control coatings of non-bioactive C₁₆-V₂A2E₂ (PA2) and C₁₆—V₂A2E₄GIKVAV (PA3), which has been previously reported to be less dynamic than PA due to the peptide sequence having more rigid and hydrophobic amino acids (V₂A2), were tested. The incorporation of PS2 increased neurite length on all PA coatings, but PA+PS2 showed the longest neurites in all conditions (FIG. 8). This suggests a synergistic effect between PS2 and the bioactive PA, where PS2 boosts the effect on neural bioactivity.

Protein levels were assessed via Western blot to assess neural maturity. Enhanced expression of the maturation markers microtubule associated protein-2 (MAP-2), post-synaptic density-95 (PSD-95), and synaptophysin (Syn) was observed in PA+PEDOT-S/OH cultures (FIG. 6e-h). Next, the functional properties of the neurons at DIV10 was investigated using single cell patch clamp. Sharper action potentials with higher amplitudes and lower resting membrane potentials in PA coatings that incorporated PEDOT-S/OH were observed (FIG. 6i-k). These electrophysiology results demonstrate that the addition of PEDOT-S/OH induces more hyperpolarized cells with stronger action potentials that are indicative of formation of more mature and active neural networks. Finally, RNA sequencing of cell cultures at DIV5 was performed to see if there were any trends in the gene profile. Gene ontology terms associated with cell adhesion, tube morphogenesis, and cell biogenesis and growth (e.g. neuronal development) were upregulated in cells cultured on PA+PEDOT-S/OH compared to PA alone, demonstrating that the conducting polymer aided with neural maturation (FIG. 71).

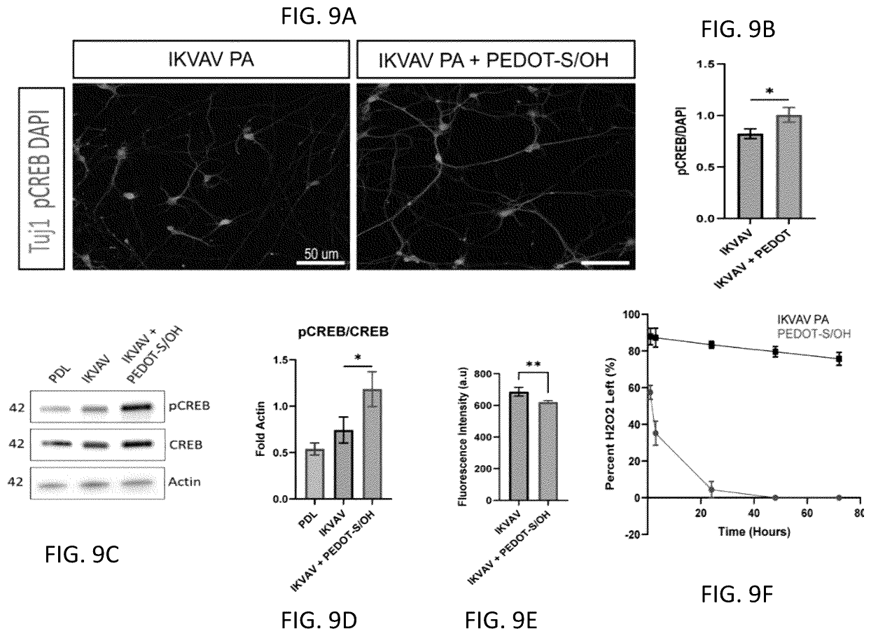

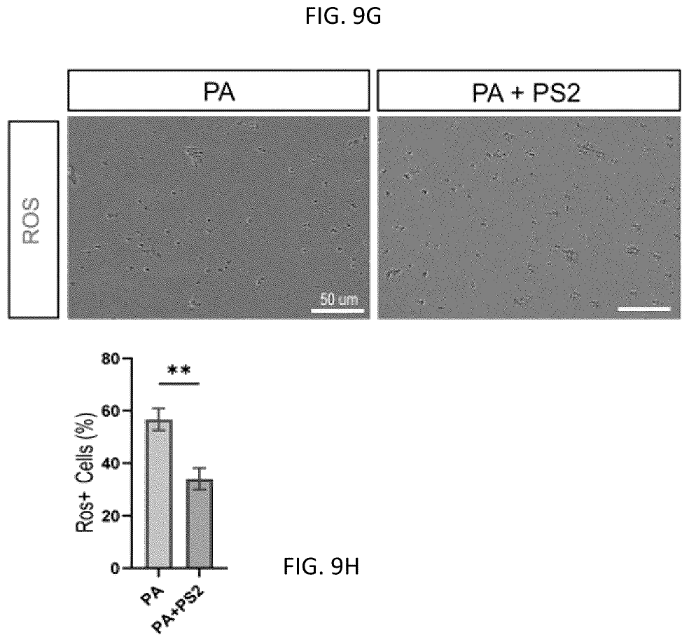

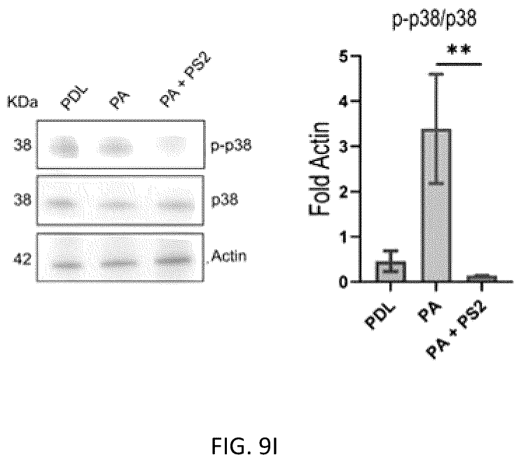

A series of protein expression experiments was performed to investigate the origin of enhanced maturation in neural cell cultures on the PEDOT-S/OH+PA material. One important transcription factor involved in neural development is cAMP response element-binding protein (CREB). When activated, it leads to a cascade of gene activation leading to increases in vital growth factors for neural growth such as brain derived nerve factor (BDNF) and VGF. In addition, CREB activation has been linked to enhancements in synaptic plasticity and cognition. Compared to IKVAV PA alone, neurons cultured on IKVAV PA+PEDOT-S/OH showed higher expression of phosphorylated CREB (pCREB) per cell (FIG. 9a, b), and higher levels of pCREB relative to total CREB which was constant across all conditions (FIG. 9c, d).

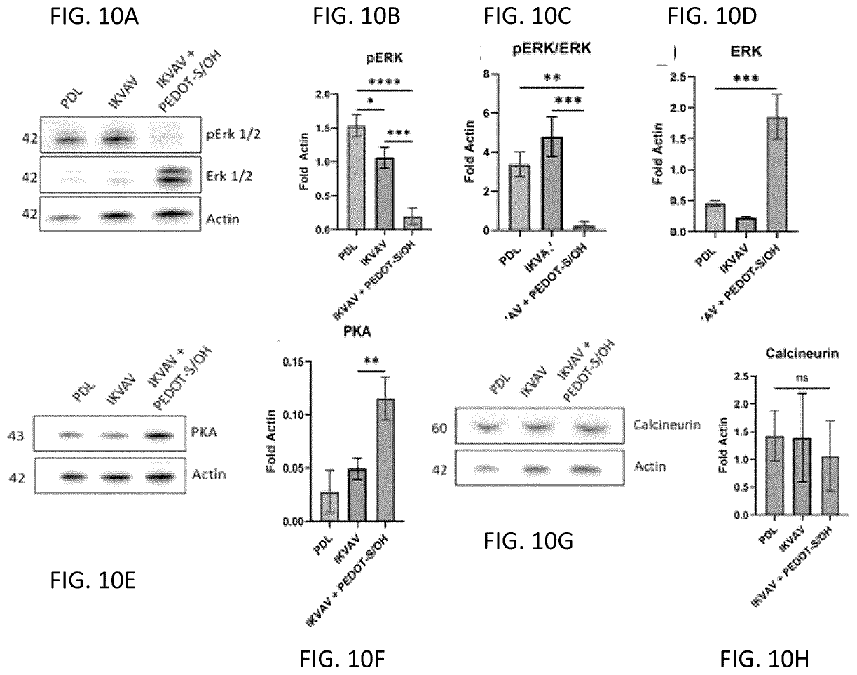

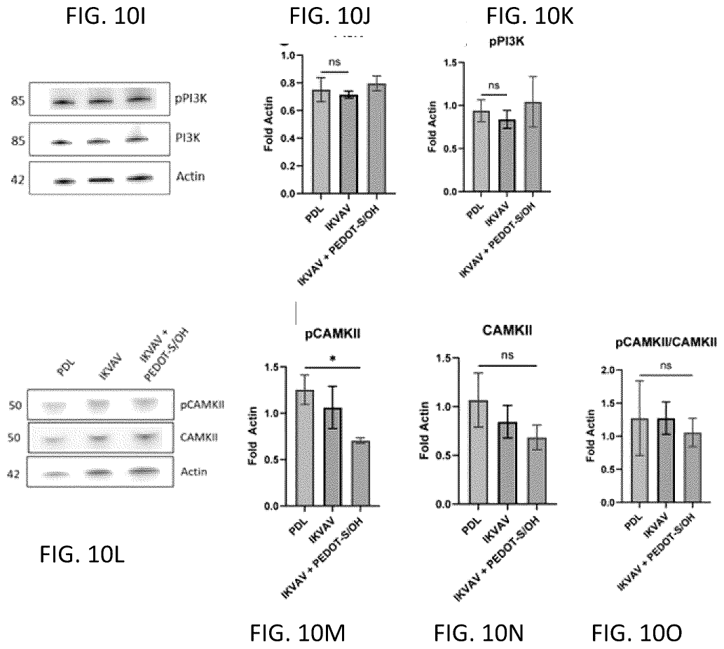

Since CREB is activated through various upstream molecular pathways, such as MAPK, cAMP, and calcium dependent pathways, protein expression of these upstream signals was next evaluated (FIG. 10). Phosphoinositide 3-kinases (P1I3K) and extracellular signal-regulated kinase (ERK 1/2) within the MAPK pathway, protein kinase A (PKA) within the cAMP pathway and calcineurin and calcium-calmodulin-dependent protein kinase II (CAMKII) within the calcium channel pathway were investigated. The addition of PEDOT-S/OH to IKVAV PA did not have any significant impact on PI3K, calcineurin or CAMKII expression (FIG. 10h). Downregulation of phosphorylated ERK 1/2 was observed (FIG. 10a-c) while total levels were upregulated (FIG. 10d), indicating an overall downregulation with the addition of PEDOT-S/OH. Upregulation of PKA in cells cultured on PEDOT-S/OH was observed (FIG. 10f), which is expected since PKA is known to downregulate ERK through competitive binding to Ras. This behavior is consistent with more mature neurons that have formed synapses. These results suggest that PS2 enhanced CREB activation through a cAMP dependent pathway, with a potential role in oxidative stress mediation.

To further elucidate the mechanism for enhanced neural maturation when neurons were cultured on the conductive substrate, the effect of the conducting polymer on reactive oxygen species (ROS) present in cell cultures was investigated. Interestingly, a significant decrease in the amount of ROS present in the media of neurons cultured on the PA+PEDOT-S/OH compared to PA alone was observed (FIG. 9e, g). Similarly, fluorescence micrographs showed lower ROS in cells on PA+PS2 coatings compared to those on PA alone (FIG. 9h). It was hypothesized that the PEDOT could act as a reducing agent of ROS species generated during cell maturation. This antioxidant effect could explain enhanced maturation of neural cells cultured in the presence of PEDOT-S/OH. To test this hypothesis and validate that the effect was attributed to the conducting polymer, hydrogen peroxide solutions were prepared in the presence of either PA and PEDOT-S/OH, the peroxide remaining over time was measured using a ROS absorbance assay. After 1 day nearly all of the peroxide was removed in the PEDOT-S/OH solution but that ˜75% of the peroxide was still present in the PA solution even after 3 days (FIG. 9f). This clearly demonstrated that the PEDOT was responsible for the reduction of hydrogen peroxide in solution, and supported the theory that the conducting polymer could enhance bioactivity of PA hydrogels by reducing oxidative stress that typically inhibits neural maturation. To complement these experiments, a Western blot was performed for p38, a protein that is phosphorylated in the presence of oxidative stress (FIG. 9I). PA+PS2 samples displayed significantly lower phosphorylated p38 compared to PA alone. These studies demonstrated that the incorporation of PS2 decreases the presence of ROS in solution as well as in the presence of cells. Thus, it was hypothesized that PS2 may act as a ROS scavenger to decrease oxidative stress in neural systems, resulting in a healthier cell environment, and allowing neurons to develop more quickly. This results in increased maturation and neural development through CREB-activated pathways.

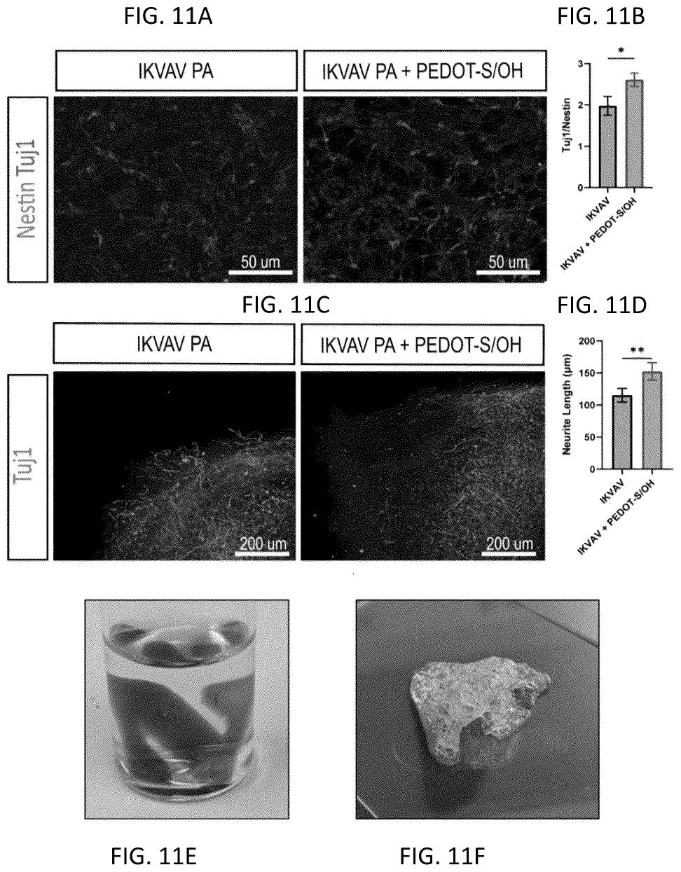

To see if the conducting polymer-PA hybrid material also enhanced bioactivity of human cells, human induced pluripotent stem cell (iPSC) derived neurons were cultured on PA+PEDOT-S/OH coatings. Increased expression of differentiated neurons (Tuj1) vs. neural progenitor cells (Nestin) on the conductive coatings was observed, indicative of an enhanced neuronal culture in human cells (FIG. 11a, b). Next, the PA+PEDOT-S/OH material was applied to the culture medium of human spinal cord organoids to determine if this effect was reproduced in a 3D model. Interestingly, sprouting of long neurites from the organoid surface in the presence of the PEDOT-S/OH was observed (FIG. 11c, d). These results demonstrate that translation of the material to human cells and therapies is feasible. Finally, the PA, PEDOT-S/OH, and GG components were combined to form a robust, 3D printable, conductive, and bioactive hydrogel. Compared to PEDOT-PA materials alone, the addition of GG allowed for facile printing of the material in arbitrary shapes (FIG. 11e, f).

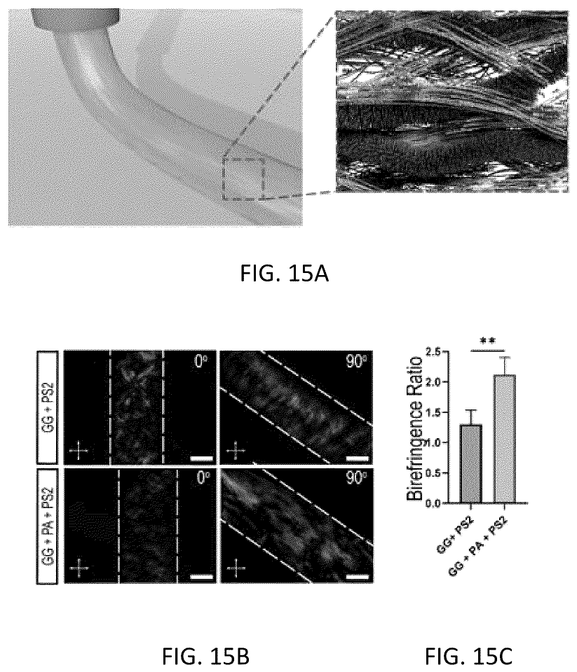

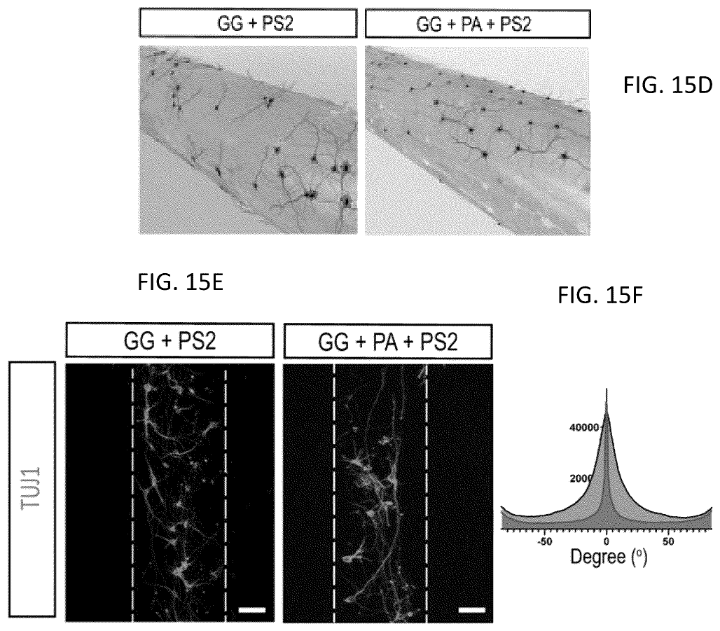

Finally, we tested the printability of these therapies by incorporating GG into the system to form a robust, conductive, and bioactive hydrogel. Printing of PAs has been previously seen to enhance the alignment of nanofibers and cells within the scaffolds.³⁸ This approach shows potential as an implantable material for the treatment of SCI to provide an aligned scaffold and promote cell regeneration across the lesion while also minimizing ROS related immune responses. Gels were printed using the BioX 3D printer to form filaments which can be used to template the alignment of cells (FIG. 15A). When PA was incorporated into GG+PS2 inks, the resulting filaments showed enhanced birefringence compared to GG+PS2 alone (FIG. 15B, 15C), which may be attributed to the shear alignment during printing of the liquid crystalline PA nanofibers. Mouse cortical neurons seeded on GG+PA+PS2 gels showed enhanced neural alignment compared to the GG+PS2 gels after 7 days in culture, highlighting the ability of the hybrid PA scaffold to direct cell outgrowth (FIG. 15D-F). Thus, this aligned scaffold may direct neurons to regenerate across the injury site.

Provided herein is a bioactive, conductive, and printable scaffold for neuronal maturation. The incorporation of the conducting polymer enhanced the conductivity of the substrate without affecting PA self-assembly. Both mouse and human derived neurons cultured on the system showed enhanced neurite extension while maintaining biocompatibility. Furthermore, the cellular pathways affected by the incorporation of the conducting polymer were examined through biochemical and genetic analysis and determined ROS reduction as a potential target. Without wishing to be bound by theory, it is possible that PS2 acts as a reactive oxidative species scavenger and promotes a more mature and electroactive neural system, allowing for enhanced neural maturation through CREB-related pathways. Moreover, PS2 maintains its effect on neural maturation in human 2D and 3D systems, enhancing neural progenitor differentiation, and organoid axon extension. These conductive, biomimetic, and printable assemblies may be used as therapies to treat injuries or disease in the central nervous system. The scaffold could be printed into CNS injury sites to promote recovery by stimulating neuron growth and synaptogenesis, while “cleaning up” excessive ROS from the environment.

### Example 2

To explore the use of permanent positive charges into the polymer backbone, which could create dense anion clouds that could be used to synergistically improve neural repair, small organic cations were incorporated into the EDOT-structure (FIG. 16). These cationic derivatives may also be used to electrostatically complex with negatively charged covalent or supramolecular scaffolds. The modified EDOT monomers could be polymerized into a homopolymer or co-polymerized with other EDOT monomers to introduce multiple functional groups into the same polymer chain.

Exemplary cationic EDOT derivatives with extended linker moieties are shown in FIG. 16. The slow reactivity of EDOT-ImCl in a chemical polymerization procedure is most likely due to the close proximity of the electron withdrawing group (the N-alkylated imidazolium ion, ImCl group) to the dioxane ring unit, resulting in electron deficient alfa positions in the thiophene. Since the EDOT-ImCl monomer was unreactive under normal conditions for chemical polymerization like sodium persulfate (NaPS) (2 eq.) and iron(III) chloride (0.1 eq.) in Milli-Q water two novel analogues (EDOT-Bu-ImCl, EDOT-EG2-ImCl) were explored where the cation is positioned over larger bond lengths from the EDOT-structure using linkerology. Two different types of linkers were explored (see Scheme 1), hydrophobic butyl-based linker, and one hydrophilic linker based on diethylene glycol. The nature of the linkers will be used to fine-tune the interactions chemical environment, and the clouds of ions around the organic imidazolium cation.

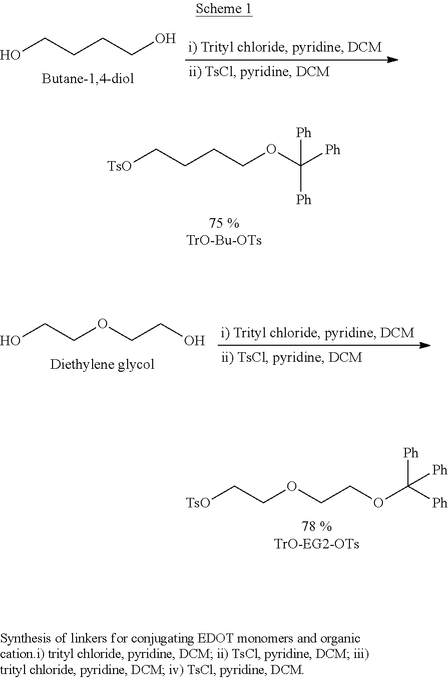 [SMILES: OCCCCO ; Cc1ccc(S(=O)(=O)OCCCCOC(c2ccccc2)(c2ccccc2)c2ccccc2)cc1 ; OCCOCCO ; Cc1ccc(S(=O)(=O)OCCOCCOC(c2ccccc2)(c2ccccc2)c2ccccc2)cc1]

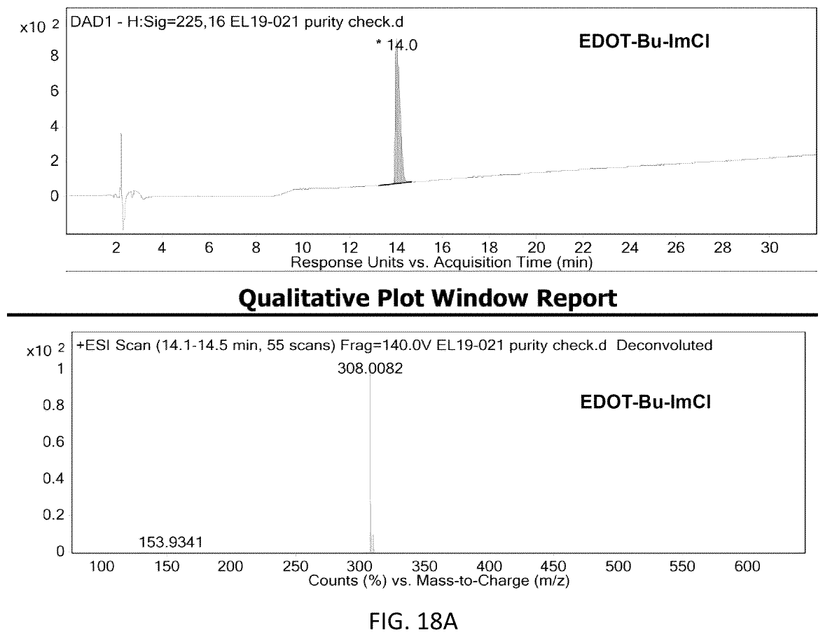

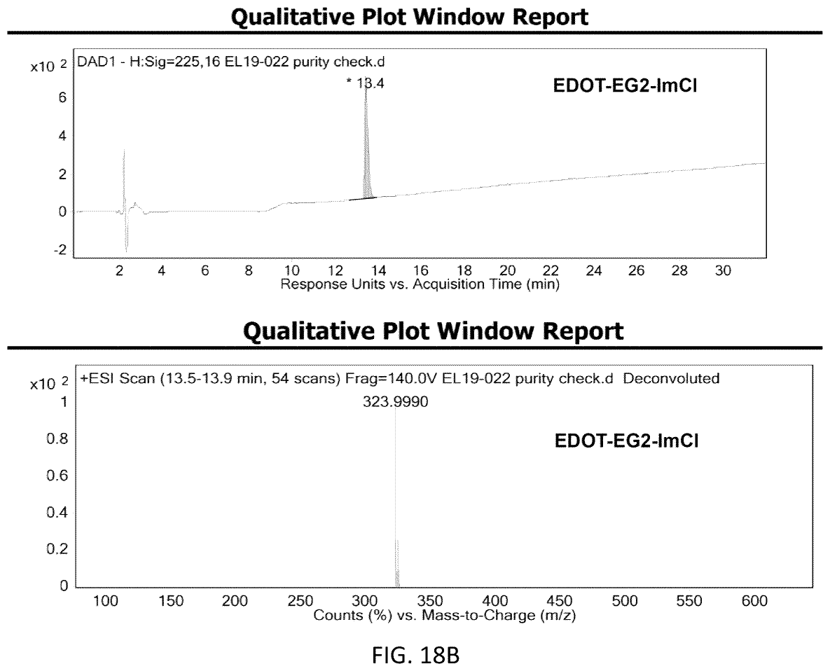

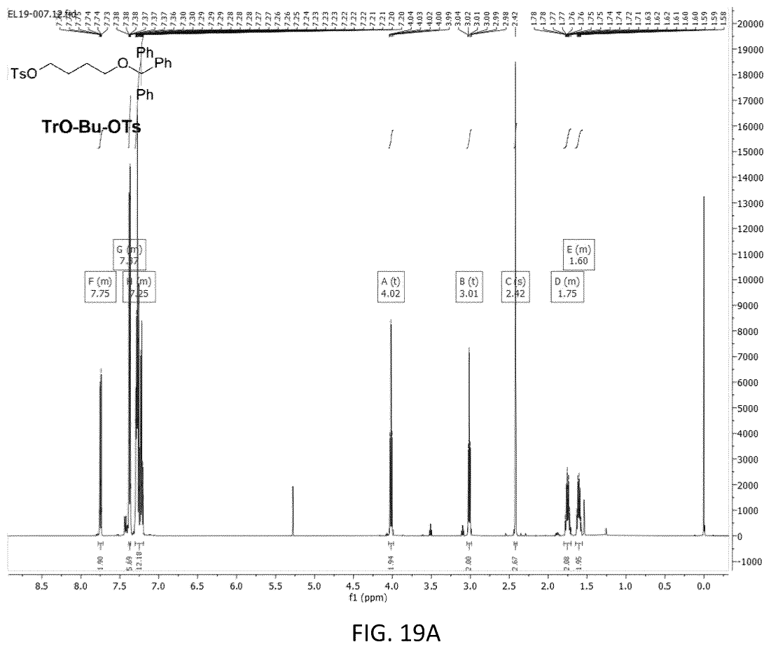

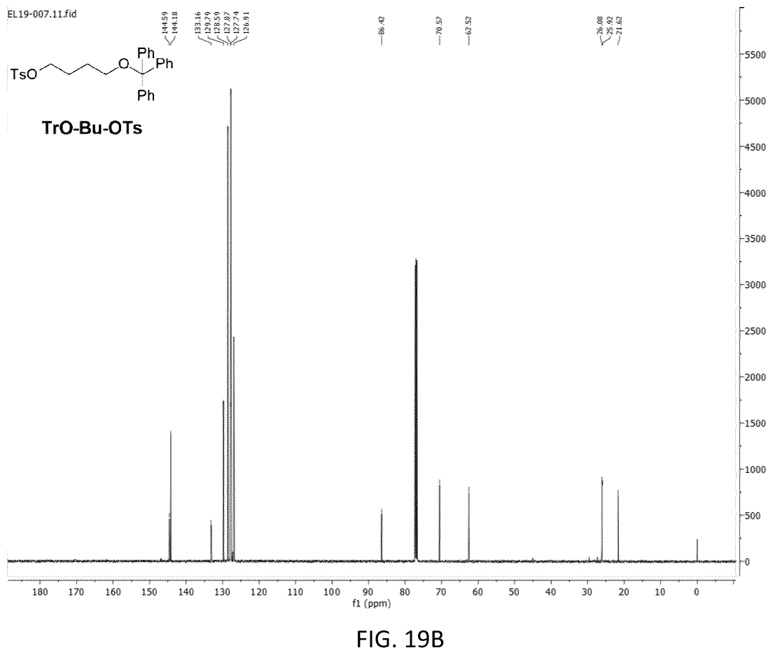

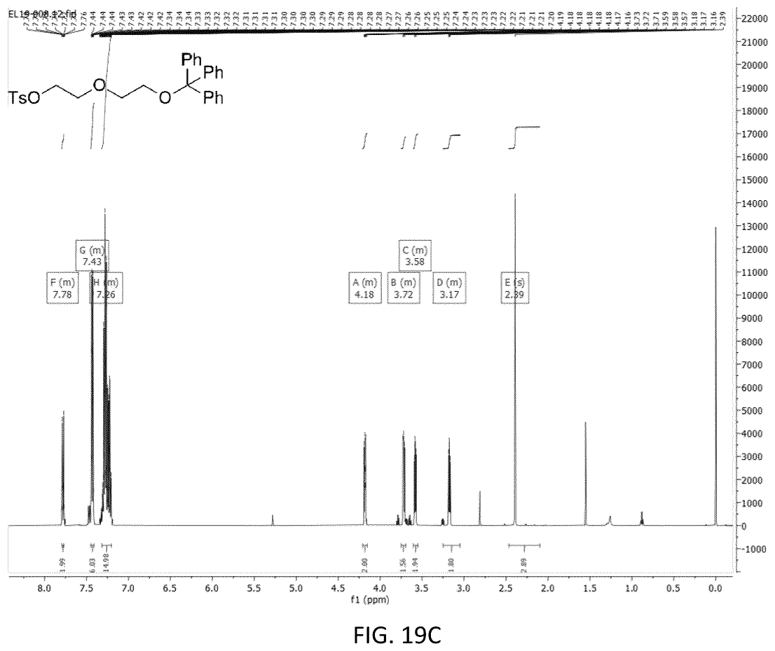

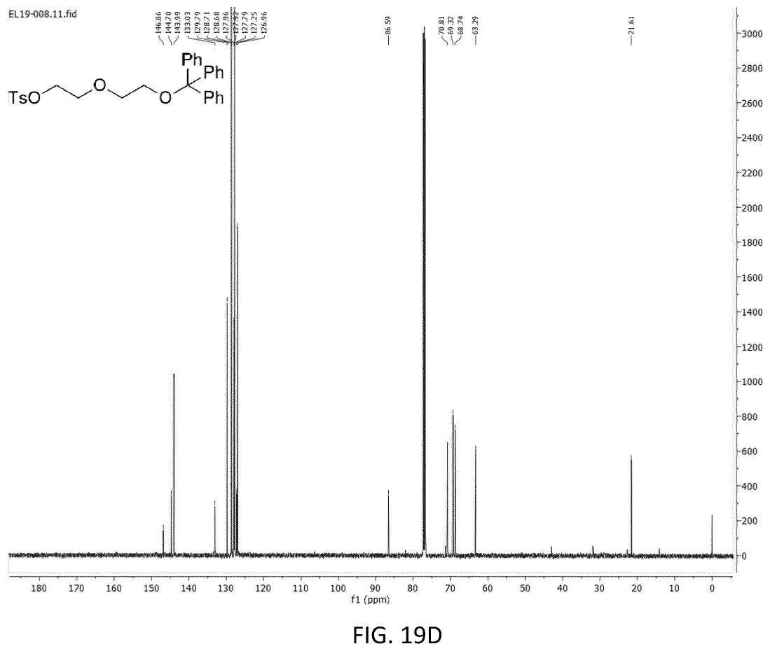

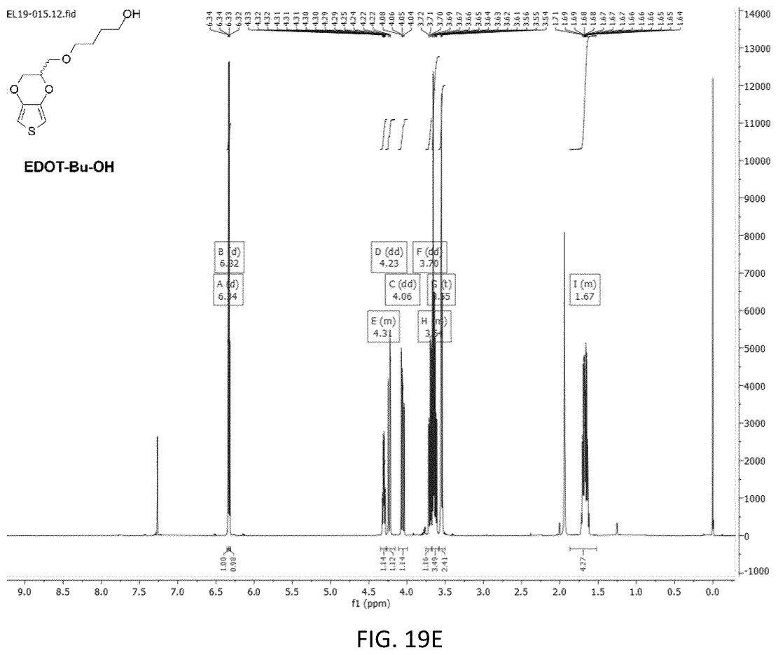

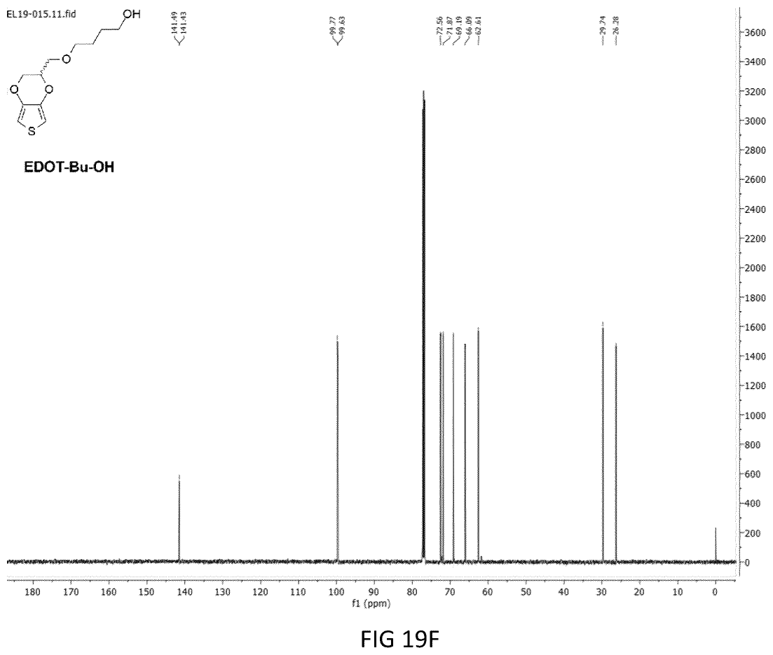

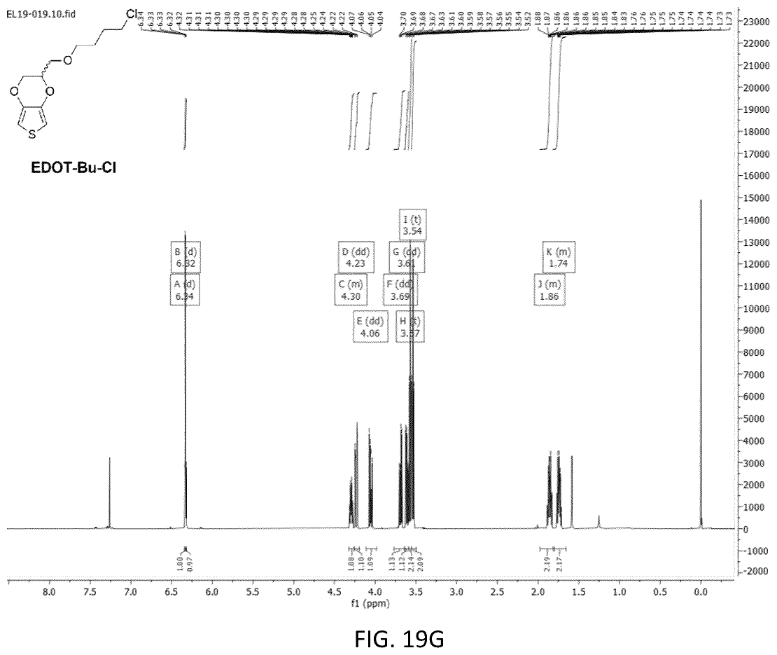

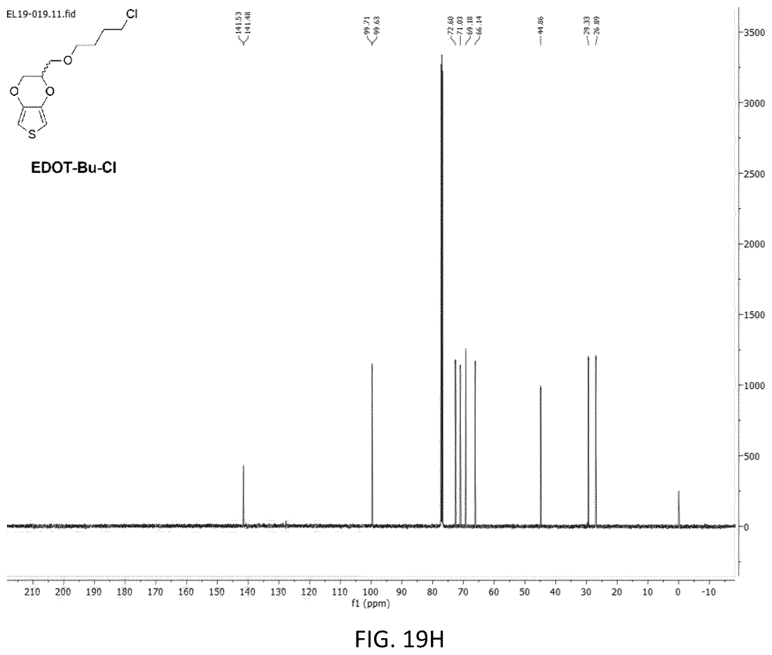

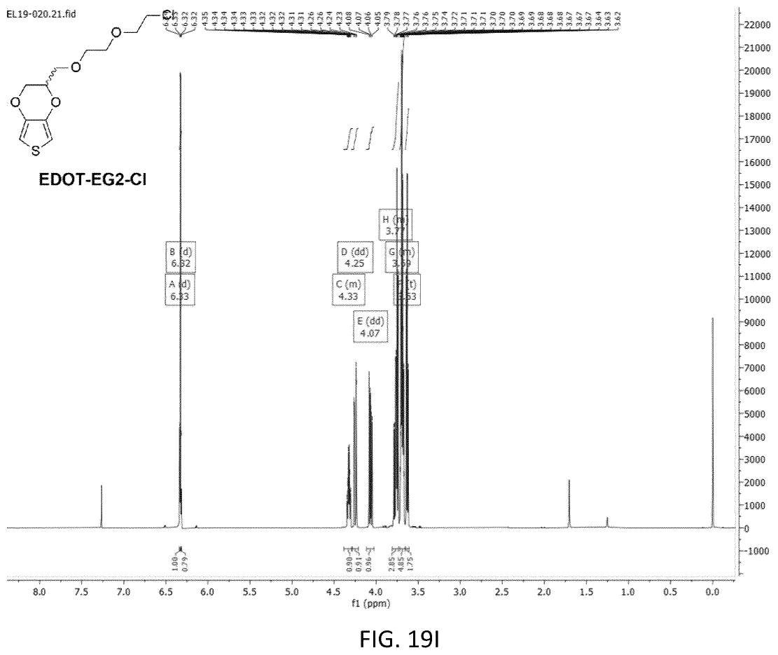

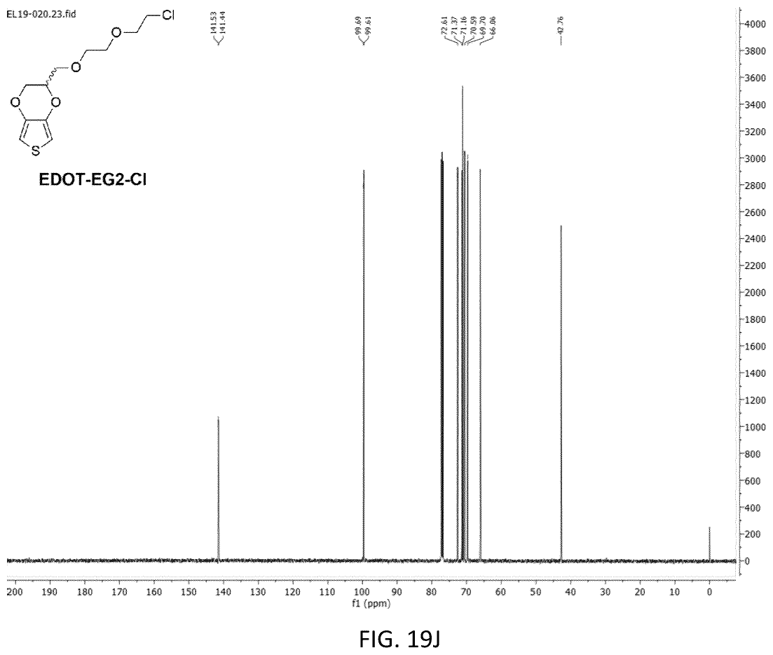

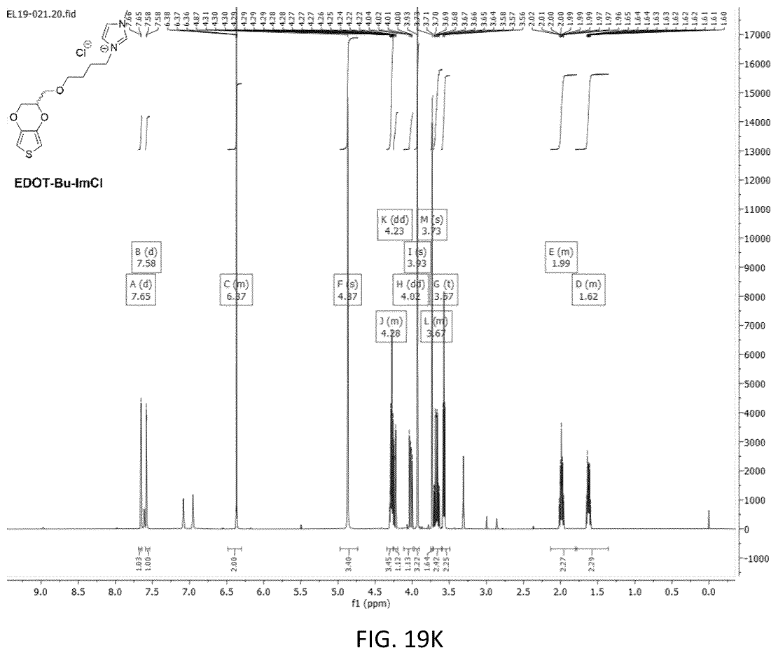

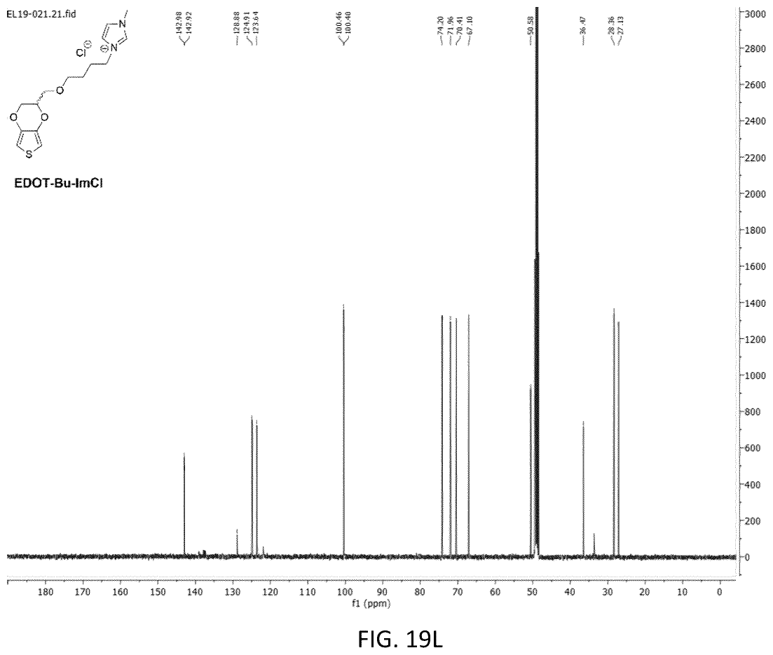

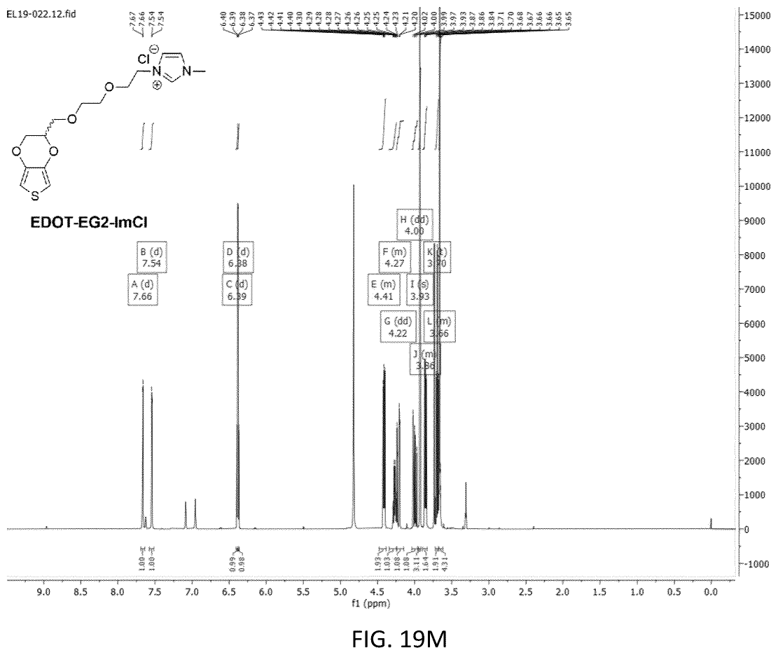

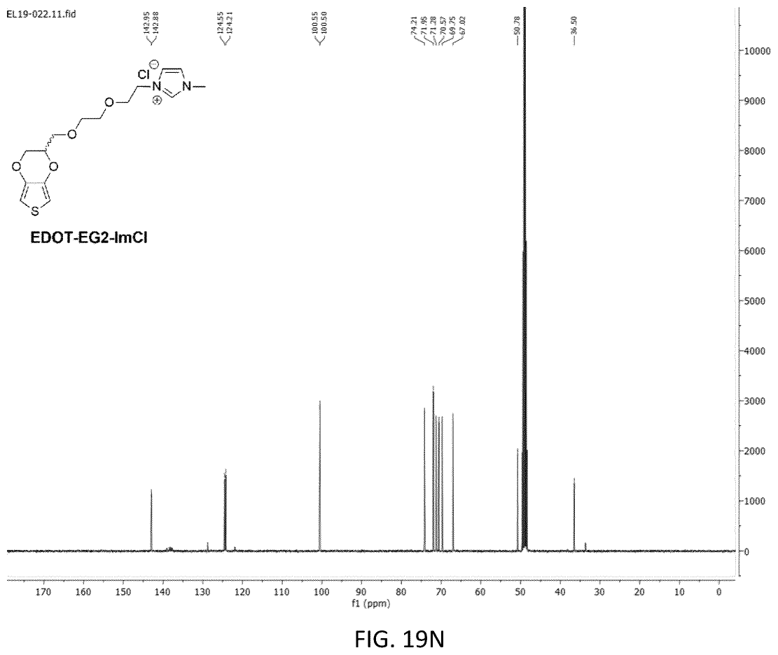

The two linker precursors TrO-Bu-OTs and TrO-EG2-OTs were conjugated to the EDOT-OH scaffold using a substitution reaction. Since initial experiments failed using an activated tosylated/mesylated EDOT-OH species or EDOT-halide as the electrophile most likely due to elimination reaction, EDOT-OH was instead used as the nucleophile in an alkylating reaction under basic conditions. The two heterobifunctional liker molecules TrO-Bu-OTs and TrO-EG2-OTs were both synthesized in two steps using a mono-protection followed by a conversion of the remaining —OH group to a O-tosyl group. The structure of TrO-Bu-OTs was confirmed using ¹H-NMR (FIG. 19A) and ¹³C NMR (FIG. 19B). The structure of TrO-EG2-OTs was confirmed using ¹H-NMR (FIG. 19C) and ¹³C NMR (FIG. 19D). The linkers were conjugated to the EDOT-OH using NaH in DMF followed by an acid catalyzed deprotection of the trityl protecting group generating EDOT-Bu-OH and EDOT-EG2-OH in 37% and 68% yield respectively over two steps. The structure of EDOT-Bu-OH was confirmed using ¹H-NMR (FIG. 19E) and ¹³C NMR (FIG. 19F). The conversion to the EDOT-halides EDOT-Bu-Cl and EDOT-EG2-Cl was performed in a two steps manner where the alcohols were activated with tosyl-groups followed by displacement using TBAC in DMF, yields 47% and 41% respectively. The structure of EDOT-Bu-Cl was confirmed using ¹H-NMR (FIG. 19G) and ¹³C NMR (FIG. 19H). The structure of EDOT-EG2-Cl was confirmed using ¹H-NMR (FIG. 19I) and ¹³C NMR (FIG. 19J). The final step introducing the terminal imidazolium ion was conducted by heating EDOT-Bu-Cl and EDOT-EG2-Cl with N-methylimidazole in DMF to generate the target compounds EDOT-Bu-ImCl and EDOT-EG2-ImCl in 61% and 54% yield respectively (Scheme 2). Purity was analyzed using LCMS and confirmed for EDOT-Bu-ImCl (FIG. 18A) and EDOT-EG2-ImCl (FIG. 18B). The structure of EDOT-Bu-ImCl was confirmed using ¹H-NMR (FIG. 19K) and ¹³C NMR (FIG. 19L). The structure of EDOT-EG2-ImCl was confirmed using ¹H-NMR (FIG. 19M) and ¹³C NMR (FIG. 19N).

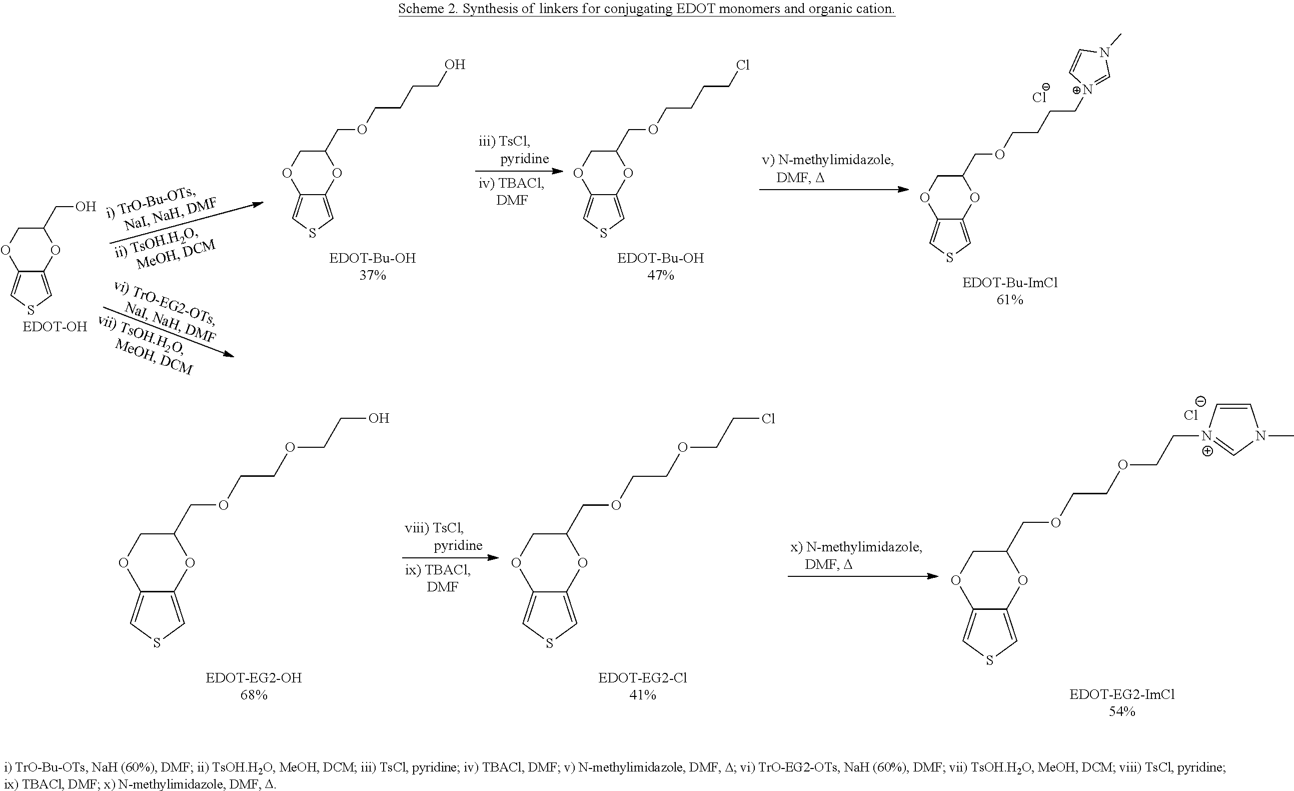

### Materials and Methods

All reagents and chemicals were purchased form Millipore Sigma, if not otherwise stated. N,N-Dimethylformamide (DMF) was dried with pre-activated 4 Å MS. Thin layer chromatography (TLC) was carried out on Merck 60 F₂₅₄ plates, reaction was monitored with UV and developed with 5% H₂SO₄ in ethanol, followed by heating at ˜−250° C. Flash column chromatography (FC) was carried out on silica gel Merck 60 (40-63 μm). Reverse-phase column chromatography (RP) was performed using LiChroprep© RP-18 (40-63 μm). Dichloromethane (DCM) was dried with pre-activated 4 Å MS.

### Experimental

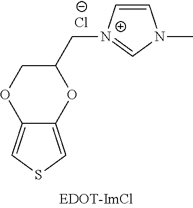 [SMILES: Cn1cc[n+](CC2COc3cscc3O2)c1 ; [Cl-]]

### EDOT-ImCl

2-Chloromethyl EDOT (EDOT-Cl) (1.50 g, 7.87 mmol) was added to N-methylimidazole (3.14 mL, 3.23 g, 39.34 mmol) and heated to 80° C. After 3 days the solution was cooled down and the mixture was evaporated and co-concentrated with xylenes. The crude was washed with n-heptane (3×20 mL). RP (95:5 Water/ACN->50:50 Water/ACN) gave EDOT-ImCl (1.87 g, 6.86, 87%) as an off-white liquid. NMR spectra in accordance with previously published data.¹ ESI-MS: [M+H]⁺ calcd for C₁₁H₁₃N₂O₂S, 237.0698; found 237.0700.

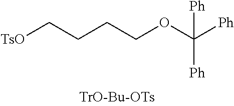 [SMILES: Cc1ccc(S(=O)(=O)OCCCCOC(c2ccccc2)(c2ccccc2)c2ccccc2)cc1]

### 4-(Trityloxy)butyl 4-methylbenzenesulfonate (TrO-Bu-OTs)²

Trityl chloride (25.00 g, 89.68 mmol) was added in portions at room temperature to a solution of 1,4-butandiol (158.94 mL, 161.64 g, 1.79 mol), pyridine (14.45 mL, 14.19 g, 179.36 mmol) in DCM (300 mL) and left to stir overnight. The reaction mixture was washed with water, NaCl (sat.aq.), 1M HCl, NaHCO₃ (sat.aq.), water, dried over MgSO₄(s), filtered and evaporated gave the crude 4-(triphenylmethoxy)-1-butanol as a white solid. R_{f}=0.68 (1:1 toluene:EtOAc). The crude intermediate was added in portions to solution of dry DCM (300 mL) and pyridine (14.45 mL, 14.19 g, 179.36 mmol). Tosyl chloride (TsCl) (25.65 g, 134.52 mmol) was added in portions at room temperature. After 30 mins additional pyridine (28.89 mL, 28.37 g, 358.72 mmol) and TsCl (17.10 g, 89.68 mmol) was added at room temperature and the reaction was stirred overnight. The solution was evaporated and co-concentrated with toluene. The remaining solid was dissolved in DCM and washed with 1M HCl, NaHCO₃ (sat.aq.), water, dried over MgSO₄(s), filtered and evaporated. FC (DCM) gave the titled compound TrO-Bu-OTs as a white solid (32.57 g, 66.93 mmol, 75% yield over two steps). R_{f}=0.91 (1:1 toluene:EtOAc). R_{f}=0.45 (DCM). NMR: ¹³C NMR (125 MHz, CDCl3) δ 144.6, 144.2, 133.2, 129.8, 128.6, 127.9, 127.7, 126.9, 86.4, 70.6, 62.5, 26.1, 25.9, 21.6; ¹H NMR (500 MHz, CDCl₃) δ 7.78-7.72 (m, 2H), 7.39-7.36 (m, 5H), 7.30-7.20 (m, 12H), 4.02 (t, J=6.5 Hz, 2H), 3.01 (t, J=6.1 Hz, 2H), 2.42 (s, 3H), 1.80-1.71 (m, 2H), 1.65-1.56 (m, 2H).

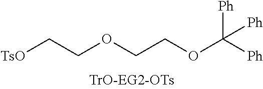 [SMILES: Cc1ccc(S(=O)(=O)OCCOCCOC(c2ccccc2)(c2ccccc2)c2ccccc2)cc1]

### 2-(2-(trityloxy)ethoxy)ethyl 4-methylbenzenesulfonate (TrO-EG2-OTs)

Trityl chloride (25.00 g, 89.68 mmol) was added in portions at room temperature to a solution of diethylene glycol (169.94 mL, 190.33 g, 1.79 mol), pyridine (14.45 mL, 14.19 g, 179.36 mmol) in DCM (300 mL) and left to stir overnight. The reaction mixture was washed with water, 1M HCl, NaHCO₃ (sat.aq.), water, dried over MgSO₄(s), filtered and evaporated gave the crude 2-(2-(trityloxy)ethoxy)ethanol as a white solid. R_{f}=0.63 (1:1 toluene:EtOAc). The crude intermediate was added in portions to solution of dry DCM (300 mL) and pyridine (43.34 mL, 42.56 g, 538.08 mmol). Tosyl chloride (TsCl) (42.56 g, 224.20 mmol) was added in portions at room temperature and the reaction was stirred overnight. The solution was evaporated and co-concentrated with toluene. The remaining solid was dissolved in DCM and washed with 1M HCl, NaHCO₃ (sat.aq.), water, dried over MgSO₄(s), filtered and evaporated. FC (DCM/n-heptane 1:1) gave the titled compound TrO-EG2-OTs as a white solid (34.96 g, 69.56 mmol, 78% over two steps). R_{f}=0.71 (DCM). NMR: ¹³C NMR (125 MHz, CDCl₃) δ 146.9, 144.7, 144.0, 133.0, 129.8, 128.7, 128.7, 128.0, 127.9, 127.8, 127.3, 127.0, 86.6, 70.8, 69.3, 68.7, 63.3, 21.6; ¹H NMR (500 MHz, CDCl₃) δ 7.79-7.77 (m, 2H), 7.45-7.41 (m, 5H), 7.32-7.20 (m, 12H), 4.21-4.15 (m, 2H), 3.75-3.69 (m, 2H), 3.61-3.55 (m, 2H), 3.25-3.05 (m, 2H), 2.39 (s, 3H). ESI-MS: [M+NH4]⁺ calcd for C₃₀H₃₀O₅S, 520.2152; found 520.2136.

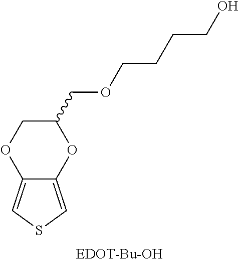 [SMILES: OCCCCOCC1COc2cscc2O1]

### 4-((2,3-dihydrothieno[3,4-b][1,4]dioxin-2-yl)methoxy)butan-1-ol (EDOT-Bu-OH)

EDOT-OH (0.50 g, 2.90 mmol) was dissolved in DMF (20 mL). NaH (60% dispersed in oil) (76.65 mg, 127 mg) was slowly added in portions at room temperature. TrO-Bu-OTs (2.12 g, 4.36 mmol) was added in one portion and the reaction was stirred at room temperature. After 1 h, NaI (435 mg, 2.90 mmol) was added and stirred overnight. The reaction was quenched with MeOH (1 mL) and the mixture was evaporated. The crude was dissolved in DCM and washed with NaCl (sat.aq.), dried over MgSO₄(s), filtered and evaporated. R_{f}=0.68 (9:1 toluene/EtOAc. Without further purification, the crude was dissolved in DCM/MeOH 2:5 (70 mL) and TsOH·H₂O (55 mg, 0.29 mmol). After 2 hours the solution was concentrated. FC (9:1 toluene/EtOAc->1:1 toluene/EtOAc) gave EDOT-Bu-OH as a colorless liquid (368 mg, 1.5 mmol, 37% yield over two steps). R_{f}=0.1 (9:1 toluene/EtOAc). NMR: ¹³C NMR (125 MHz, CDCl₃) δ 141.5, 141.4, 99.8, 99.6, 72.6, 71.9, 69.2, 66.1, 62.6, 29.7, 26.3; ¹H NMR (500 MHz, CDCl₃) δ 6.34 (d, J=3.7 Hz, 1H), 6.32 (d, J=3.6 Hz, 1H), 4.35-4.26 (m, 1H), 4.23 (dd, J=11.7, 2.2 Hz, 1H), 4.06 (dd, J=11.7, 7.4 Hz, 1H), 3.70 (dd, J=10.5, 5.2 Hz, 1H), 3.67-3.58 (m, 3H), 3.55 (t, J=5.9 Hz, 2H), 1.87-1.52 (m, 4H). ESI-MS: [M+Na]⁺ calcd for C₁₁H₁₆O₄S, 267.0667; found 267.0675.

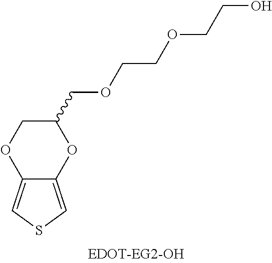 [SMILES: OCCOCCOCC1COc2cscc2O1]

### 2-(2-((2,3-dihydrothieno[3,4-b][1,4]dioxin-2-yl)methoxy)ethoxy)ethanol (EDOT-EG2-OH)

EDOT-OH (0.50 g, 2.90 mmol) was dissolved in DMF (20 mL). NaH (60% dispersed in oil) (76.65 mg, 127 mg) was slowly added in portions at room temperature. TrO-Bu-OTs (2.12 g, 4.36 mmol) was added in one portion and the reaction was stirred at room temperature. After 30 mins, NaI (435 mg, 2.90 mmol) was added and stirred 3 hours. The reaction was quenched with MeOH (1 mL) and the mixture was evaporated. The crude was dissolved in DCM and washed with NaCl (sat.aq.), dried over MgSO₄(s), filtered and evaporated. Rf=0.50 (9:1 toluene/EtOAc. Without further purification, the crude was dissolved in DCM/MeOH 2:5 (70 mL) and TsOH·H₂O (55 mg, 0.29 mmol). After 2 hours the solution was concentrated. FC (9:1 toluene/EtOAc->1:1 toluene/EtOAc) gave EDOT-EG2-OH as a colorless liquid (515 mg, 1.98 mmol, 68% yield over two steps). Rf=0.1 (9:1 toluene/EtOAc). NMR: ¹³C NMR (125 MHz, CDCl₃) δ 141.5, 141.4, 99.8, 99.7, 72.6, 72.5, 71.2, 70.3, 69.6, 66.0, 61.8; ¹H NMR (500 MHz, CDCl₃) δ 6.34 (d, J=3.7 Hz, 1H), 6.33 (d, J=3.7 Hz, 1H), 4.38-4.27 (m, 1H), 4.24 (dd, J=11.7, 2.3 Hz, 1H), 4.21-4.17 (m, 1H), 4.07 (dd, J=11.7, 7.3 Hz, 1H), 3.77 (dd, J=10.6, 5.2 Hz, 1H), 3.75-3.72 (m, 1H), 3.72-3.66 (m, 4H), 3.64-3.58 (m, 2H), 3.55-3.48 (m, 1H).

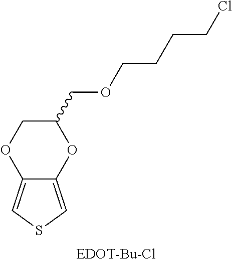 [SMILES: ClCCCCOCC1COc2cscc2O1]

### 2-((4-chlorobutoxy)methyl)-2,3-dihydrothieno[3,4-b][1,4]dioxine (EDOT-Bu-Cl)

EDOT-Bu-OH (341 mg, 1.40 mmol) was dissolved in pyridine (5.0 mL, 62.07 mmol) whereupon TsCl (399 mg, 2.09 mmol) was added and stirred at room temperature overnight. The reaction mixture was evaporated and co-concentrated with toluene. The residue was dissolved in DCM and washed with 1M HCl, NaHCO₃ (sat.aq.), dried over MgSO₄(s), filtered and concentrated to afford the crude tosylated EDOT-Bu-OH. Rf=0.80 (1:1 toluene/EtOAc). Without further purification the tosylated compound was dissolved in DMF (5 mL) and tetrabutylammonium chloride (TBAC) (583 mg, 2.10 mmol) was added. After 1 hour, the reaction mixture was evaporated and co-concentrated with xylenes. FC (toluene->9:1 toluene/EtOAc) gave the titled compound EDOT-Bu-Cl as a colorless liquid (171 mg, 0.65 mmol, 47% over two steps). Rf=0.74 (9:1 toluene/EtOAc). NMR: ¹³C NMR (125 MHz, CDCl₃) δ 141.5, 141.5, 99.7, 99.6, 72.6, 71.0, 69.2, 66.1, 44.9, 29.3, 26.9; ¹H NMR (500 MHz, CDCl₃) δ 6.34 (d, J=3.7 Hz, 1H), 6.32 (d, J=3.7 Hz, 1H), 4.32-4.26 (m, 1H), 4.23 (dd, J=11.6, 2.2 Hz, 1H), 4.06 (dd, J=11.7, 7.4 Hz, 1H), 3.69 (dd, J=10.4, 5.0 Hz, 1H), 3.61 (dd, J=10.4, 5.8 Hz, 1H), 3.57 (t, J=6.5 Hz, 2H), 3.54 (t, J=6.2 Hz, 2H), 1.98-1.80 (m, 2H), 1.82-1.66 (m, 2H).

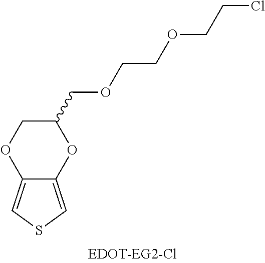 [SMILES: ClCCOCCOCC1COc2cscc2O1]

### 2-((2-(2-chloroethoxy)ethoxy)methyl)-2,3-dihydrothieno[3,4-b][1,4]dioxine (EDOT-EG2-Cl)

EDOT-EG2-OH (647 mg, 2.49 mmol) was dissolved in pyridine (10 mL, 124 mmol) whereupon TsCl (710 mg, 3.73 mmol) was added and stirred at room temperature overnight. The reaction mixture was evaporated and co-concentrated with toluene. The residue was dissolved in DCM and washed with 1M HCl, NaHCO₃ (sat.aq.), dried over MgSO₄(s), filtered and concentrated to afford the crude tosylated EDOT-EG2-OH. Rf=0.68 (1:1 toluene/EtOAc). Without further purification the tosylated compound was dissolved in DMF (10 mL) and tetrabutylammonium chloride (TBAC) (1.04 g, 3.73 mmol) was added. After 1 hour, the reaction mixture was evaporated and co-concentrated with xylenes. FC (toluene->9:1 toluene/EtOAc) gave the titled compound EDOT-EG2-Cl as a colorless liquid (282 mg, 1.01 mmol, 41% over two steps). Rf=0.57 (9:1 toluene/EtOAc). NMR: ¹³C NMR (125 MHz, CDCl₃) δ 141.5, 141.4, 99.7, 99.6, 72.6, 71.4, 71.2, 70.6, 69.7, 66.1, 42.8; ¹H NMR (500 MHz, CDCl₃) δ 6.33 (d, J=3.7 Hz, 1H), 6.32 (d, J=3.7 Hz, 1H), 4.39-4.28 (m, 1H), 4.25 (dd, J=11.7, 2.3 Hz, 1H), 4.07 (dd, J=11.6, 7.5 Hz, 1H), 3.81-3.73 (m, 3H), 3.72-3.66 (m, 5H), 3.63 (t, J=5.8 Hz, 2H). ESI-MS: [M+H]⁺ calcd for C₁₁H₁₅CO₄S, 279.0458; found 279.0465.

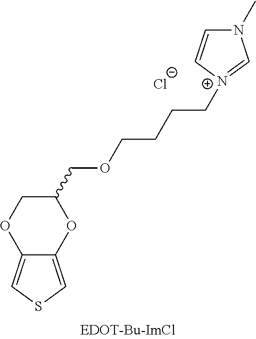 [SMILES: Cn1cc[n+](CCCCOCC2COc3cscc3O2)c1 ; [Cl-]]

### 3-(4-((2,3-dihydrothieno[3,4-b][1,4]dioxin-2-yl)methoxy)butyl)-1-methyl-1H-imidazol-3-ium chloride (EDOT-Bu-ImCl)

To a stirred solution of EDOT-Bu-Cl (150 mg, 0.57 mmol) in DMF (5 mL), N-methylimidazole (351 mg, 4.28 mmol) was added and the mixture was heated to 80° C. overnight. The solution was cooled down and evaporated and co-concentrated with xylenes. FC (7:4:1 DCM/MeOH/H₂O) gave EDOT-Bu-ImCl as a colorless oil (121 mg, 0.35 mmol, 61% yield). R_{f}=0.64 (7:4:1 DCM/MeOH/H₂O). NMR: ¹³C NMR (125 MHz, MeOD) δ 143.0, 142.9, 128.9, 124.9, 123.6, 100.5, 100.4, 74.2, 72.0, 70.4, 67.1, 50.6, 36.5, 28.4, 27.1; ¹H NMR (500 MHz, MeOD) δ 7.65 (d, J=2.0 Hz, 1H), 7.58 (d, J=2.0 Hz, 1H), 6.48-6.30 (m, 2H), 4.34-4.26 (m, 3H), 4.23 (dd, J=11.7, 2.3 Hz, 1H), 4.02 (dd, J=11.7, 7.4 Hz, 1H), 3.93 (s, 3H), 3.71-3.60 (m, 2H), 3.57 (t, J=6.0 Hz, 2H), 2.13-1.78 (m, 2H), 1.80-1.35 (m, 2H). (H-2 imidazolium proton not detected due to NMR solvent). ESI-MS: [M]⁺ calcd for C₁₅H₂₁N₂O₃S, 309.1267; found 309.1273.

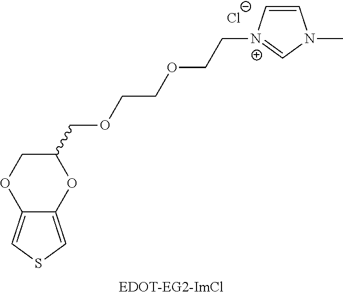 [SMILES: Cn1cc[n+](CCOCCOCC2COc3cscc3O2)c1 ; [Cl-]]

### 3-(2-(2-((2,3-dihydrothieno[3,4-b][1,4]dioxin-2-yl)methoxy)ethoxy)ethyl)-1-methyl-1H-imidazol-3-ium chloride (EDOT-EG2-ImCl)

To a stirred solution of EDOT-EG2-Cl (250 mg, 0.89 mmol) in DMF (5 mL), N-methylimidazole (536 mg, 6.73 mmol) was added and the mixture was heated to 80° C. overnight. The solution was cooled down and evaporated and co-concentrated with xylenes. FC (7:4:1 DCM/MeOH/H₂O) gave EDOT-EG2-ImCl as a colorless oil (174 mg, 0.48 mmol, 54% yield). R_{f}=0.63 (7:4:1 DCM/MeOH/H₂O). NMR: ¹³C NMR (100 MHz, CD₃OD) δ 142.95, 142.88, 124.55, 124.21, 100.55, 100.50, 74.21, 71.95, 71.28, 70.57, 69.75, 67.02, 50.78, 36.50; ¹H NMR (400 MHz, CD₃OD) δ 7.66 (d, J=2.0 Hz, 1H), 7.54 (d, J=2.0 Hz, 1H), 6.39 (d, J=3.6 Hz, 1H), 6.38 (d, J=3.6 Hz, 1H), 4.48-4.39 (m, 2H), 4.34-4.24 (m, 1H), 4.22 (dd, J=11.7, 2.3 Hz, 1H), 4.00 (dd, J=11.7, 7.4 Hz, 1H), 3.93 (s, 3H), 3.89-3.83 (m, 2H), 3.70 (t, J=5.2 Hz, 2H), 3.68-3.62 (m, 4H). (H-2 imidazolium proton not detected due to NMR solvent). ESI-MS: [M]⁺ calcd for C₁₅H₂₁N₂O₄S, 325.1217; found 325.1207.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

1. A copolymer comprising (i) at least one sulfonatoalkoxy EDOT monomer or at least one cationic EDOT monomer, and (ii) and at least one heterocyclic monomer.

2. The copolymer of claim 1, wherein the at least one sulfonatoalkoxy EDOT monomer comprises EDOT-S or wherein the at least one cationic EDOT monomer comprises an EDOT imidazolium monomer.

3. (canceled)

4. The copolymer of claim 1, wherein the at least one heterocyclic monomer comprises at least one thiophene monomer.

5. The copolymer of claim 4, wherein the at least one heterocyclic monomer comprises at least one EDOT monomer.

6. The copolymer of claim 5, wherein the at least one EDOT monomer is functionalized with a hydroxyl group or a N-methylimidazole group.

7. The copolymer of claim 1, wherein the at least one heterocyclic monomer is an EDOT-OH monomer.

8. The copolymer of claim 1, comprising the at least one sulfonatoalkoxy EDOT monomer or at least one cationic EDOT monomer, and the least one heterocyclic monomer at a ratio of 1:10 to 10:1.

9. The copolymer of claim 8, comprising the at least one sulfonatoalkoxy EDOT monomer or at least one cationic EDOT monomer, and the at least one heterocyclic monomer at a ratio of 4:1.

10. A composition comprising the copolymer of claim 1 and a hydrogel material.

11. The composition of claim 10, wherein the copolymer is directly linked to the hydrogel material, or wherein the composition further comprises a linker that covalently or noncovalently links the copolymer to the hydrogel material.

12. The composition of claim 11, wherein leakage of the copolymer from the hydrogel is less than 10%, less than 5%, less than 2%, or less than 1% over the course of 7 days.

13. The composition of claim 1, wherein the hydrogel material comprises a polysaccharide.

14. The composition of claim 10, wherein the hydrogel material comprises a plurality of bioactive peptide amphiphiles, each bioactive peptide amphiphile comprising a hydrophobic tail, a structural peptide segment, a charged peptide segment, and a bioactive moiety.

15. The composition of claim 14, wherein the hydrophobic tail comprises an 8-24 carbon alkyl chain (C₈₋₂₄), the structural peptide segment has a total propensity for forming β-sheet conformation of 4 or less, the charged peptide segment comprises EE, EEE, or EEEE (SEQ ID NO: 3), the bioactive moiety comprises IKVAV (SEQ ID NO: 1), and/or the bioactive moiety is attached to the charged peptide segment by a linker.

16. (canceled)

17. The composition of claim 15, wherein the structural peptide segment comprises A₂G₂(SEQ ID NO: 2).

18. (canceled)

19. (canceled)

20. (canceled)

21. The composition of claim 15, wherein the linker is a single glycine (G) residue.

22. The composition of claim 14, wherein the composition comprises a plurality of bioactive peptide amphiphiles comprising C₈₋₂₄-A₂G₂E₄GIKVAV (SEQ ID NO: 11).

23. The composition of claim 14, further comprising one or more filler peptide amphiphiles, wherein the filler peptide amphiphiles comprise a hydrophobic non-peptide tail, a structural peptide segment, and a charged peptide segment, and do not comprise a bioactive moiety.

24. A method comprising contacting a cell or a subject with the composition of claim 10.

25. (canceled)

26. A system comprising a cell cultured on the composition of claim 10.

27. (canceled)

28. (canceled)


SEQUENCE LISTING (11 sequences)

SEQ ID NO: 1 - protein, 5 aa, synthetic construct
IKVAV

SEQ ID NO: 2 - protein, 4 aa, synthetic construct
AAGG

SEQ ID NO: 3 - protein, 4 aa, synthetic construct
EEEE

SEQ ID NO: 4 - protein, 4 aa, synthetic construct
VVAA

SEQ ID NO: 5 - protein, 6 aa, synthetic construct
VVVAAA

SEQ ID NO: 6 - protein, 4 aa, synthetic construct
AAVV

SEQ ID NO: 7 - protein, 6 aa, synthetic construct
AAAVVV

SEQ ID NO: 8 - protein, 6 aa, synthetic construct
AAAGGG

SEQ ID NO: 9 - protein, 5 aa, synthetic construct
VEVAA

SEQ ID NO: 10 - protein, 9 aa, synthetic construct
VVVAAAEEE

SEQ ID NO: 11 - protein, 14 aa, synthetic construct
AAGGEEEEGIKVAV